(19) United States
(12) Patent Application Publication
(10) Pub. No.: US 2010/0056468 A1
(43) Pub. Date: Mar. 4, 2010

(54) Pyrimidine Derivatives As Anticancer Agents

(75) Inventors: Lakshmi P. Kotra, Greensboro, NC (US); Emil F. Pai, Toronto (CA); Christopher J. Paige, Toronto (CA); Angelica M. Bello, Toronto (CA)

Correspondence Address: Pabst Patent Group LLP, 1545 PEACHTREE STREET NE, SUITE 320, ATLANTA, GA, 30309, US

(73) Assignee: University Health Network, Toronto (CA)

(21) Appl. No.: 12/522,511

(22) PCT Filed: Jan. 8, 2008
(86) PCT No.: PCT/CA08/00014

§ 371 Date: Nov. 4, 2009

Related U.S. Application Data
(60) Provisional application No. 60/883,919, filed on Jan. 8, 2007.

(51) Int. Cl. A61K 31/706; C07H 19/10; C07H 19/056; A61P 35/00

(52) U.S. Cl. 514/49; 536/26.8; 536/28.5; 514/52

(57) ABSTRACT

The present invention includes methods of treating or preventing cancer by administering an effective amount of 6-substituted pyrimidine derivatives of the Formula I to a subject need thereof:

### FIELD OF THE INVENTION

The present invention relates to methods of using certain 6-substituted pyrimidine derivatives for the treatment of cancer.

### BACKGROUND OF THE INVENTION

ODCase (EC 4.1.1.23) plays a central role in the de novo synthesis of the ribonucleotide, uridine 5′-O-monophosphate (UMP) from orotidine 5′-O-monophosphate (OMP). This enzyme catalyzes the decarboxylation of OMP to UMP (final step in FIG. 1A). UMP is a building block, synthesized de novo from aspartic acid, for the synthesis of pyrimidine nucleotides such as uridine 5′-triphosphate (UTP), cytidine 5′-triphosphate (CTP), thymidine 5′-triphosphate (TTP) and 2′-deoxycytidine 5′-triphosphate (dCTP) (FIG. 1B). These pyrimidine nucleotides are the building blocks for the synthesis of nucleic acids for cell replication and survival. ODCase exhibits an extraordinary level of catalytic rate enhancement of over 17 orders of magnitude compared to the uncatalyzed decarboxylation reaction in water at neutral pH 7.0 at 25° C.^{i,ii,iii,iv} An interesting difference when one looks at this enzyme across species is that in certain higher level organisms such as human, rat or mouse, ODCase is a part of the bifunctional enzyme, UMP synthase.^{v}

Over the past three decades, various studies focused on developing inhibitors against ODCase with little understanding of the mechanism of decarboxylation, and most of those efforts did not result in effective inhibitors. Such investigations targeting ODCase focused on developing inhibitors against cancer, malaria, and RNA viral infections. 6-Aza-UMP, 6-hydroxy-UMP (or BMP), pyrazofurin 5′-monophosphate, xanthosine 5′-monophosphate (XMP) and 6-thiocarboxamido-UMP, the structures of which are shown in FIG. 2 are some of the potent inhibitors that have been studied against ODCase.^{i,vi,vii,viii,ix,x} Development of these inhibitors into clinically useful drugs has been limited due to their toxicities, pharmacokinetics and lack of specificity (vide infra).^{viii} There are only limited structure-activity relationship and drug design studies against ODCase. Thus, ODCase did not gain much traction in 1980s and 1990s as a drug target.

If one carefully analyzes the biochemical and pharmacological basis, ODCase is a fascinating enzyme as a drug target. For example, Plasmodia species such as P. falciparum and P. vivax are dependent on their own de novo synthesis of pyrimidine nucleotides due to the absence of the salvage pathway in these parasites.^{xi} Selective inhibition of plasmodial ODCase has been proposed as a strategy to design compounds directed against malaria and a limited number of orotate analogs have been investigated as potential drugs against the malarial parasite.^{vi,xii,xiii} ODCase has also been identified as a target for drugs directed against RNA viruses like pox and flavi viruses; the former causing increasing concern as a potential bioterrorist weapon.^{xiv,xv,xvi,xvii} In humans, pyrimidine nucleotides are synthesized via both the de novo and salvage pathways (FIG. 1B).^{xviii} A few inhibitors of ODCase such as 6-azauridine and pyrazofurin exhibited good anticancer activities against a number of clinical tumor models.^{xix,xx} These studies and the potential role of ODCase in cell survival and replication make this a unique, yet untapped drug target.

Aside from its pharmacological interest, ODCase has been a favorite enzyme for biochemists and structural biologists due to its unusual catalytic properties. A number of mechanisms for the decarboxylation of OMP by ODCase were proposed prior to and after the availability of X-ray crystal structures for ODCases.^{xxi,xxii,xxiii,xxiv} Although ideas of covalent catalysis were discussed, the plausible mechanisms do not seem to support a covalent species formation as a key step during decarboxylation by ODCase. A structural analysis of the catalytic site of ODCase from Methanobacterium thermoautotrophicum (Mt) revealed two aspartate residues (Asp70 and Asp75B, the latter contributed by the second subunit of the dimeric ODCase) and two lysine residues (Lys42 and Lys72) that are held via a network of strong hydrogen bonds (FIG. 3A).^{ii,xxii,xxv,xxvi,xxvii} These residues are proposed to exert strong steric and electrostatic stress onto the C-6 carboxylate group of OMP and eliminate the carboxyl group.^{xxii}

The x-ray crystal structures of ODCase from ten different species are known today. In 2000, four x-ray crystal structures of ODCase brought insights into the catalytic mechanism of this enzyme. Based on the structure of S. cerevisiae ODCase complexed with the transition-state analogue BMP, a transition-state stabilization mechanism of OMP decarboxylation was proposed.^{xxiv} A similar proposal was also suggested by Appleby et al. based on the crystal structure of ODCase (Bacillus subtilis) complexed with the product, UMP.^{xxiii} These authors suggested that the decarboxylation reaction proceeds via an electrophilic substitution in which C-6 is protonated by Lys62 as the carbon dioxide molecule is released.^{xxiii} The structure of the ODCase enzyme from E. coli co-crystallized with BMP was the basis of the proposal submitted by Harris et al.^{xxviii} Based on the proximity of the carboxylate moiety on OMP and Asp71 residue in the active site of ODCase, it was proposed that OMP decarboxylation depends on the existence of a shared proton between Asp71 and the carboxyl group of the substrate.^{xxviii} A similar mechanism involving electrostatic repulsion was put forward by Wu et al.^{xxii} The electrostatic repulsion mechanism points to the active site aspartate residue. In all structurally known species, the location and function of this residue is highly conserved. The catalytic residues, Asp70 and Lys72 are located near the reaction center C-6 of the pyrimidine ring of the substrate OMP and Asp70 (M thermoautotrophicum) was postulated to cause electrostatic destabilization of the enzyme-substrate complex. Lys72 in the active site furnishes the proton to neutralize the carbanion developed after the departure of the carboxylate.^{xxii}

Human ODCase is the target for anticancer activity. A sequence alignment of the ODCase region of human UMP synthase and Mt ODCase revealed that overall there is 40% similarity and 26% identity between these two sequences. However, the active site of human ODCase is almost identical to that of Mt ODCase, with very few differences (FIG. 3B). For example, the loop that encloses the phosphate moiety is longer in human UMP synthase than in Mt ODCase (arrow at 11 'o clock in FIG. 3B). There is a His residue in human UMP synthase corresponding to the Gly44 residue in MT ODCase (an arrow at 5 ′o clock in FIG. 3B).

There are few reports in the literature of ODCase being targeted for anticancer drug development. 6-Aza-uracil exhibited anticancer activity in a number of tumor models.^{viii} It is anticipated that 6-aza-uracil is transformed into its mononucleotide form, 6-aza-uridine 5′-monophosphate (FIG. 2) in vivo and thus inhibits ODCase, impairing the de novo production of pyrimidine nucleotides. 6-Aza-uridine uridine 5′-monophosphate inhibits yeast and Mt ODCases with inhibition constants (Kᵢ) of 64 nM and 11 μM, respectively.^{,xxix} Another potent inhibitor of ODCase, pyrazofurin (FIG. 2) has been investigated, including in clinical trials. Pyrazofurin 5′-monophosphate, which is a C-nucleoside, has a Kᵢ of 5 nM and its nucleoside analog is readily taken up by the cells and converted into its monophosphate form.^{xxx} In a separate study, measurement of levels of pyrimidine and purine intermediates in cultured mouse L1210 leukemia cells showed that 25 μM pyrazofurin induced an eight-fold increase in the accumulation of OMP and an abrupt decrease in the pyrimidine ribosyl mononucleotides.^{xxxi} Pyrazofurin however was not clinically developed further due to its toxicity to patients in phase I studies.

### SUMMARY OF THE INVENTION

A series of C6 substituted pyrimidine nucleotides were synthesized to investigate the mechanism of decarboxylation by ODCase (FIG. 4). During the course of these investigations using structural biology, enzymology and mechanistic investigations, the corresponding nucleoside analogs were screened in a number of cancer cell lines. Based on the interesting structural features of these molecules and their anticancer activities, a novel focused library of C6 substituted pyrimidine nucleoside derivatives have been developed as potent anticancer agents.

Accordingly, the present invention includes a method of treating cancer comprising administering to a subject in need thereof of an effective amount of a compound selected from a compound of Formula I, tautomers thereof and pharmaceutically acceptable salts, solvates, and prodrugs thereof:

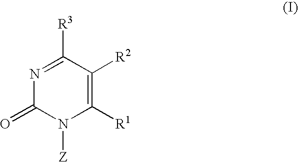

wherein,
R¹ is selected from C₁₋₆alkyl, C(O)OC₁₋₆alkyl, CN, N₃, I, Br, —CHO, —NHNH₂, —NHOH, —ONH₂, —NC, —NH₂, —NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, H, NHC(O)OC₁-C₆alkyl and NHC(O)C₁-C₆alkyl;
Z is selected from:

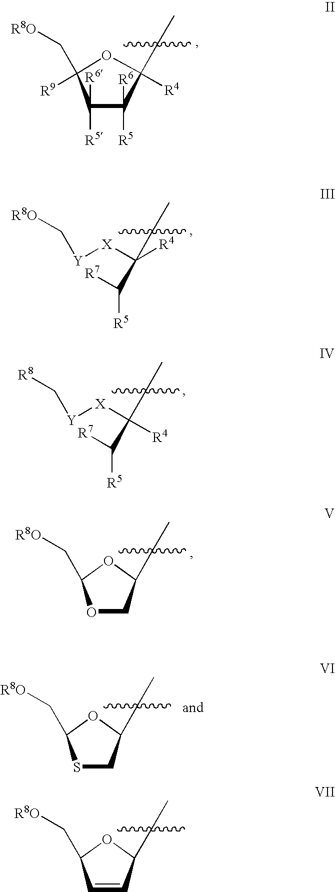

wherein,
R⁴ is selected from H, F, C₁-C₆alkyl and hydroxy-substituted-C₁-C₆alkyl;
One of R⁵ and R⁶ is selected from hydrogen and F and the other is selected from H, OH and F and one of R^{5′} and R^{6′} is selected from hydrogen and F and the other is selected from H, OH and F or R⁵ and R⁶ or R^{5′} and R^{6′} together are ═O or ═CH₂;
R⁷ is selected from H, F and OH;
R⁸ is selected from H, C(O)C₁-C₆alkyl, P(O)(OH)₂, P(O)(OC₁-C₆alkyl)₂ and P(O)(OC₁-C₆alkyl)OH;
R⁹ is selected from H, F, N₃, CN, C₁-C₆alkyl; and
X—Y is selected from —CH₂—O—, O—CH₂—, —CH₂—S— and —S—CH₂—, with the proviso that when R² is halo, R¹ is not iodo.

In further embodiments, the present invention includes a use of a compound selected from a compound of Formula I as defined above, tautomers thereof, and pharmaceutically acceptable salts, solvates, and prodrugs thereof, for the treatment of cancer, as well as a use of a compound selected from a compound of Formula I as defined above, tautomers thereof, and pharmaceutically acceptable salts, solvates, and prodrugs thereof, for the preparation of a medicament for the treatment of cancer.

Also including in the present invention is a compound selected from a compound of Formula I, tautomers thereof, and pharmaceutically acceptable salts, solvates, and prodrugs thereof, for use in treating cancer.

Recent trends in anticancer therapy are steering towards combination therapies and where possible, combining antibody-based therapies with small-molecule therapies.^{xxxii} Additionally, establishment of multi-targeted therapies by using a combination of agents targeted to several distinct enzyme molecules providing maximal impact of kill on the cancer cells is an important area of development. Thus, C6 substituted pyrimidine nucleosides, due to their complementary substitution pattern on the pyrimidine ring, will be able to afford such possibilities.

According to another aspect of the present invention, there is included a pharmaceutical composition for the treatment of cancer comprising an anti-cancer effective amount of a compound selected from a compound of Formula I as defined above, and pharmaceutically acceptable salts, solvates, and prodrugs thereof, and a pharmaceutically acceptable carrier therefore.

The present invention provides a compound of Formula Ia selected from:

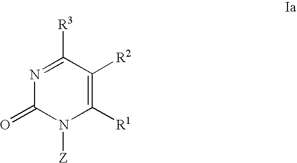

R¹is selected from CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, NHC(O)OC₁-C₆alkyl and NHC(O)C₁-C₆alkyl;
Z is selected from

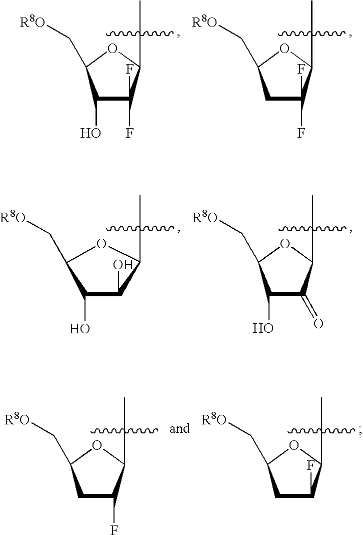

R⁸ is selected from H, C(O)C₁-C₆alkyl, P(O)(OH)₂, P(O)(OC₁-C₆alkyl)₂ and P(O)(OC₁-C₆alkyl)OH,
   tautomers thereof, and pharmaceutically acceptable salts, solvates and prodrugs thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in great detail with reference to the drawings in which:

FIG. 1A is a schematic showing the de novo biosynthesis of uridine monophosphate (UMP) from aspartic acid.

FIG. 1B is a schematic showing the de novo biosynthesis of pyrimidine nucleotides from UMP.

FIG. 2 shows the chemical structures of analogs of orotidine monophosphate (OMP) that are known as inhibitors of ODCase.

FIG. 3A is a stereo view of the active site of Methanobacterium thermoautotrophicum (Mt) ODCase (PDB code: 1DV7). Key residues Lys72, Asp70, Lys42, and Asp20 are labeled; several crystallographic waters are also shown as spheres. Enzyme backbone is shown as a ribbon color-coded according to the secondary structural elements.

FIG. 3B shows an overlap of the X-ray structure of ODCase from Mt and homology model of the ODCase portion of the human UMP synthase. Arrows point to the most obvious difference in the otherwise very similar active sites. BMP is shown in a ball-and-stick representation bound in the active site.

FIG. 4 is a bar graph showing the results of an in vivo pilot study on 70Z/3 leukemic mice (n=2). Spleen weights and the cellularity counts are reported for two doses of compound Ic.

FIG. 5 shows reversible inhibition of Hs ODCase by compound In (Table 2). (A) Thermograms representing the reaction rate in the control reaction and in the presence of various concentration of In. (B) Reaction rate at different substrate concentration in the presence of various concentration of In. Graphical representation of the determination of the reversible inhibition constant Kᵢ for the inhibition of Hs ODCase by In from (C) double-reciprocal Lineweaver-Burk plot and (D) Dixon plot.

FIG. 6 shows reversible inhibition of Hs ODCase by Iah (Table 2) (A) Thermograms representing the reaction rate in the control reaction and in the presence of various concentration of Iah. (B) Reaction rate at different substrate concentration in the presence of various concentration of Iah. Graphical representation of the determination of the reversible inhibition constant Kᵢ for the inhibition of Hs ODCase by Iah from (C) double-reciprocal Lineweaver-Burk plot and (D) Dixon plot.

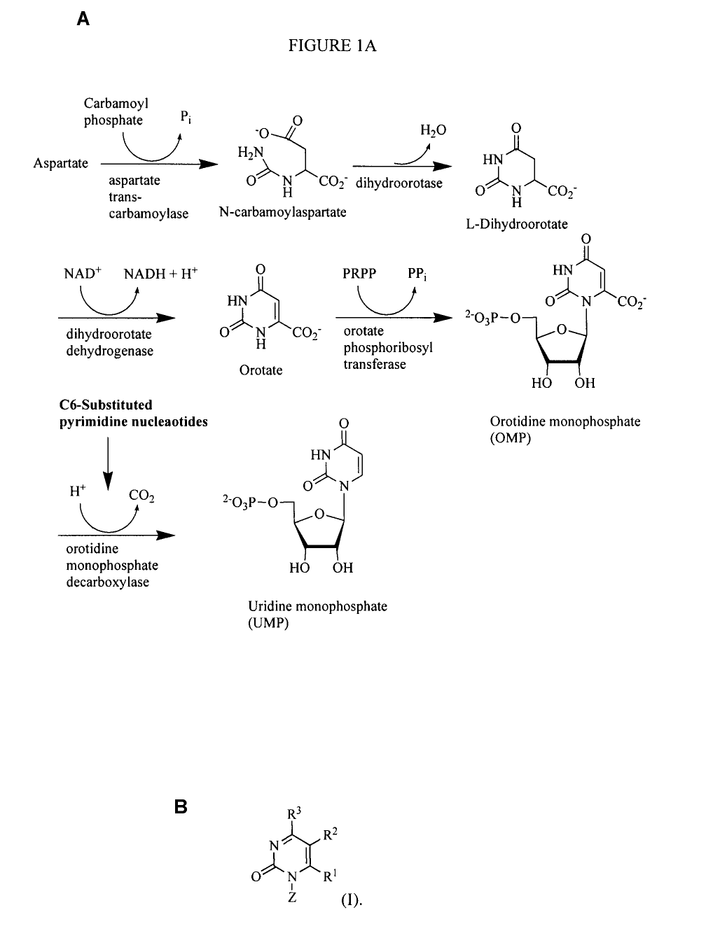

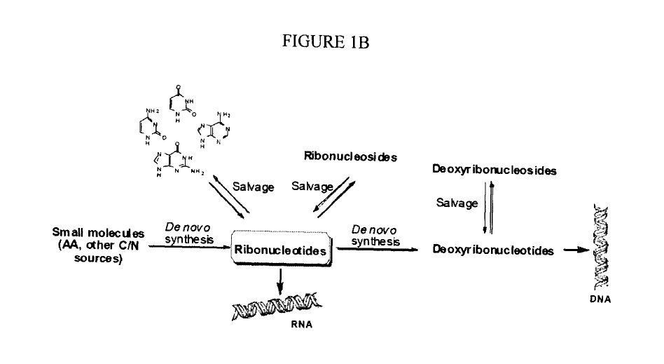

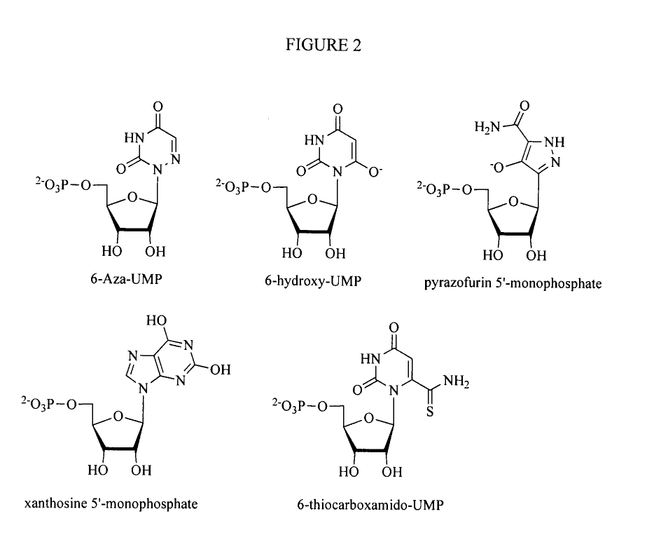

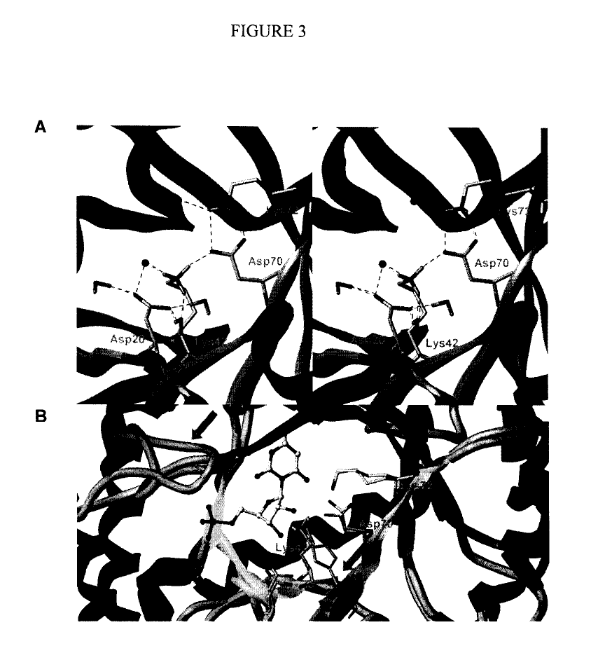

### DETAILED DESCRIPTION OF THE INVENTION

### I. Definitions

The term “C₁₋ₙalkyl” as used herein means straight and/or branched chain, saturated alkyl radicals containing from one to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl radical.

The term “fluoro-substituted C₁₋ₙalkyl” as used herein means straight and/or branched chain, saturated alkyl radicals containing from one to n carbon atoms in which one or all of the hydrogen atoms have been replaced with a fluorine, and includes (depending on the identity of “n”) trifluoromethyl, pentafluoroethyl, fluoromethyl and the like, where the variable n is an integer representing the largest number of carbon atoms in the alkyl radical.

The term “hydroxy-substituted C₁₋ₙalkyl” as used herein means straight and/or branched chain, saturated alkyl radicals containing from one to n carbon atoms in which one or two of the hydrogen atoms have been replaced with a hydroxyl group, and includes (depending on the identity of “n”) CH₂OH, CHOHCH₂CH₃, CH₂CHOHCH₂CH₂OH and the like, where the variable n is an integer representing the largest number of carbon atoms in the alkyl radical.

The term “halo” as used herein means halogen and includes chloro, fluoro, bromo and iodo.

The term “tautomer” as used herein refers to compounds that are interconvertible by a formal migration of a hydrogen atom or proton, accompanied by a switch of a single bond and an adjacent double bond. In solutions where tautomerization is possible, a chemical equilibrium of the tautomers will be reached. The exact ratio of the tautomers depends on several factors, including temperature, solvent and pH.

The term “solvate” as used herein means a compound of Formula I, or a salt of a compound of Formula I, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “compound(s) of the invention” as used herein means compound(s) of Formula I, and salts, solvates and prodrugs thereof.

The term “pharmaceutically acceptable salt” means an acid addition salt or a basic addition salt which is suitable for or compatible with the treatment of patients.

The term “pharmaceutically acceptable acid addition salt” as used herein means any non-toxic organic or inorganic salt of any base compound of the invention, or any of its intermediates. Basic compounds of the invention that may form an acid addition salt include, for example, where the R² and/or R³ is NH₂ and NHC₁₋₆alkyl. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any non-toxic organic or inorganic base addition salt of any acid compound of the invention, or any of its intermediates. Acidic compounds of the invention that may form a basic addition salt include, for example, where R⁸ is a phosphate. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art. Other non-pharmaceutically acceptable basic addition salts, may be used, for example, in the isolation of the compounds of the invention, for laboratory use, or for subsequent conversion to a pharmaceutically acceptable acid addition salt.

As mentioned earlier, ODCase synthesizes UMP de novo, which then is transformed into other ribosyl and deoxyribosyl pyrimidine nucleotides. These pyrimidine nucleotides are essential for the synthesis of new DNA and RNA for the replication of cells, especially proliferating cancer cells. ODCase inhibitors interfere with the synthesis of UMP and thus impair the production of pyrimidine nucleotides and consequently with the synthesis of RNA and DNA impairing the cell division. Cell division is faster in cancer cells than in other types of cells and this is a common characteristic of cancer cells. Accordingly, the term “cancer” as used herein refers to any cancers where there is unregulated cell division. Examples of cancer that may be treated using the compounds of the invention include, but are not limited to, skin cancer, melanoma, prostate cancer, breast cancer, colorectal cancer, ovarian cancer, leukemia and lymphoma, in particular, melanoma, ovarian cancer, breast cancer, leukemia, lymphoma, lung cancer, head and neck cancer, esophageal cancer and pancreatic cancer. While the compounds of the invention may act by modulating ODCase activity, one of skill in the art will appreciate that other modes or mechanisms of action for the compounds of the invention are possible.

The term a “therapeutically effective amount”, “effective amount” or a “sufficient amount” of a compound of the present invention is a quantity sufficient to, when administered to the subject, including a mammal, for example a human, effect beneficial or desired results, including clinical results, and, as such, an “effective amount” or synonym thereto depends upon the context in which it is being applied. For example, in the context of inhibiting ODCase, for example, it is an amount of the compound sufficient to achieve such an inhibition in ODCase activity as compared to the response obtained without administration of the compound. In the context of disease, therapeutically effective amounts of the compounds of the present invention are used to treat, modulate, attenuate, reverse, or effect cancer in a subject. An “effective amount” is intended to mean that amount of a compound that is sufficient to treat, prevent or inhibit cancer. The amount of a given compound of the present invention that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art. Also, as used herein, a “therapeutically effective amount” of a compound of the present invention is an amount which prevents, inhibits, suppresses or reduces cancer (e.g., as determined by clinical symptoms or the amount of cancer cells) in a subject as compared to a control. As defined herein, a therapeutically effective amount of a compound of the present invention may be readily determined by one of ordinary skill by routine methods known in the art.

In an embodiment, a therapeutically effective amount of a compound of the present invention ranges from about 0.1 to about 15 mg/kg body weight, suitably about 1 to about 5 mg/kg body weight, and more suitably, from about 2 to about 3 mg/kg body weight. The skilled artisan will appreciate that certain factors may influence the dosage required to effectively treat a subject, or prevent a subject, suffering from cancer and these factors include, but are not limited to, the severity of the disease or disorder, previous treatments, the general health and/or age of the subject and other diseases present.

Moreover, a “treatment” or “prevention” regime of a subject with a therapeutically effective amount of the compound of the present invention may consist of a single administration, or alternatively comprise a series of applications. For example, the compound of the present invention may be administered at least once a week. However, in another embodiment, the compound may be administered to the subject from about one time per month to about four times daily, suitably from about one time per week to about once daily, for a given treatment. The length of the treatment period depends on a variety of factors, such as the severity of the disease, the age of the patient, the concentration and the activity of the compounds of the present invention, or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

As used herein, “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Design of suitable dosing regimens are routine for one skilled in the art. In particular embodiments, two substances will be administered substantially simultaneously, i.e. within minutes of each other, or in a single composition that comprises both substances.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with cancer or manifesting a symptom associated with cancer.

To “inhibit” or “suppress” or “reduce” a function or activity, such as ODCase activity, is to reduce the function or activity when compared to otherwise same conditions except for a condition or parameter of interest, or alternatively, as compared to another conditions.

The term “subject” or “patient” or synonym thereto, as used herein includes all members of the animal kingdom, especially mammals, including human. The subject or patient is suitably a human.

### II. Methods of the Invention

The present invention includes a method of treating cancer comprising administering to a subject in need thereof an effective amount of a compound selected from a compound of Formula I, tautomers thereof, and pharmaceutically acceptable salts, solvates, and prodrugs thereof:

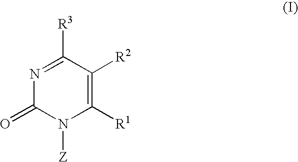

wherein,
R¹ is selected from C₁₋₆alkyl, C(O)OC₁₋₆alkyl, CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, H, NHC(O)OC₁-C₆alkyl and NHC(O)C₁-C₆alkyl;
Z is selected from:

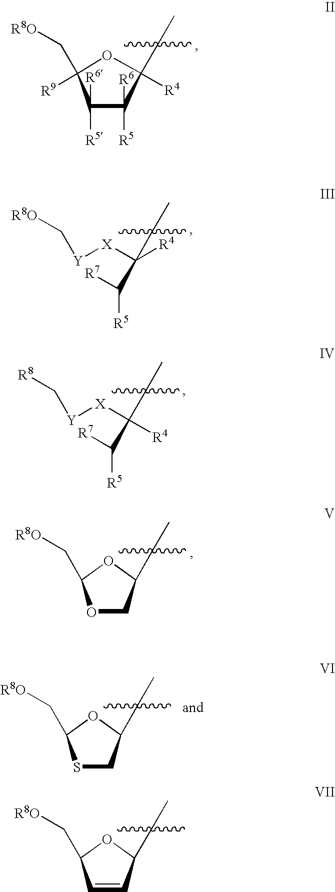

wherein,
R⁴ is selected from H, F, C₁-C₆alkyl and hydroxy-substituted-C₁-C₆alkyl;
One of R⁵ and R⁶ is selected from hydrogen and F and the other is selected from H, OH and F and one of R^{5′} and R^{6′} is selected from hydrogen and F and the other is selected from H, OH and F or R⁵ and R⁶ or R^{5′} and R^{6′} together are ═O or ═CH₂;
R⁷ is selected from H, F and OH;
R⁸ is selected from H, C(O)C₁-C₆alkyl, P(O)(OH)₂, P(O)(OC₁-C₆alkyl)₂ and P(O)(OC₁-C₆alkyl)OH;
R⁹ is selected from H, F, N₃, CN, C₁-C₆alkyl; and
X—Y is selected from —CH₂—O—, O—CH₂—, —CH₂—S— and —S—CH₂—, with the proviso that when R² is halo, R¹ is not iodo.

In the method of the invention, R¹ in the compounds of Formula I is selected from C₁₋₆alkyl, C(O)OC₁₋₆alkyl, CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂. In embodiments of the invention, R¹ in the compounds of Formula I is selected from CH₃, C(O)CH₃, C(O)CH₂CH₃, CN, N₃, I, Br, NH₂, NHCH₃, N(CH₃)₂, NHCO₂C₁₋₆alkyl, NHOH, ONH₂, C(O)NH₂. In further embodiments of the invention, R¹ in the compounds of Formula I is selected from N₃ and NH₂.

In the method of the invention, R² in the compounds of Formula I is selected H, halo, C₁-C₆alkyl, C₁-C₆alkoxy, fluoro-substituted-C₁-C₆alkyl, fluoro-substituted-C₁-C₆alkoxy, N₃, NH₂ and CN. In embodiments of the invention, R² in the compounds of Formula I is H. In further embodiments of the invention, R² in the compounds of Formula I is halo, suitably F, Br or I, more suitably F.

In the method of the invention, R³ in the compounds of Formula I is selected from OH, NH₂, H, NHC(O)OC₁-C₆alkyl and NHC(O)C₁-C₆alkyl. In embodiments of the invention, R³ in the compounds of Formula I is selected OH and NH₂. When R³ in the compounds of Formula I is selected OH and NH₂, the compounds of formula I may exist as one of the following tautomers:

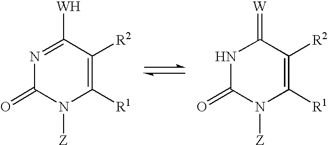

where W is O or NH. In embodiments of the invention W is O and the favoured tautomer is:

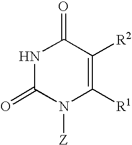

In the method of the invention, Z in the compounds of Formula I is selected from:

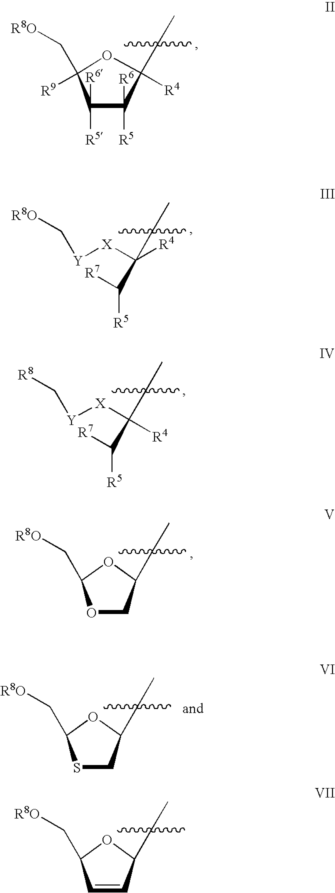

In an embodiment of the invention, Z is of the Formula II.

In the method of the invention, R⁴ in the compounds of Formula I is selected from H, F, C₁-C₆alkyl and hydroxy-substituted-C₁-C₆alkyl. In an embodiment of the invention R⁴ in the compounds of Formula I is H.

In the method of the invention, the compounds of Formula I include those where one of R⁵ and R⁶ is selected from hydrogen and F and the other is selected from H, OH and F and one of R^{5′} and R^{6′} is selected from hydrogen and F and the other is selected from H, OH and F or R⁵ and R⁶ or R^{5′} and R^{6′} together are ═O or ═CH₂. In an embodiment of the invention, R⁵ and R^{5′} are both OH and R⁶ and R^{6′} are both H. In a further embodiment of the invention, R⁵ is H, R^{5′} is OH and R⁶ and R^{6′} are both H. In yet another embodiment of the invention, R⁵ and R⁶ together are ═O and R^{5′} and R^{6′} are both H or R^{5′} is OH and R^{6′} is H. In yet another embodiment of the invention, R⁵ and R⁶ are both F and R^{5′} and R^{6′} are both H or R^{5′} is OH and R^{6′} is H. In still another embodiment of the invention R⁵ is H, R⁶ is OH, R^{5′} is OH and R^{6′} is H. In a further embodiment of the invention, one or R⁵ and R⁶ is F and the other is H and R^{5′} and R^{6′} are both H

In the method of the invention, R⁷ in the compounds of Formula I is selected from H, F and OH, suitably H or OH.

In the method of the invention, R⁸ in the compounds of Formula I is selected from H, C(O)C₁-C₆alkyl, P(O)(OH)₂, P(O)(OC₁-C₆alkyl)₂ and P(O)(OC₁-C₆alkyl)OH. In embodiments of the invention, R⁸ in the compounds of Formula I is selected from H, C(O)C₁-C₄alkyl, P(O)(OH)₂, P(O)(OC₁-C₄alkyl)₂ and P(O)(OC₁-C₄alkyl)OH. In further embodiments of the invention, R⁸ in the compounds of Formula I is selected from H, C(O)CH₃, P(O)(OH)₂, P(O)(OCH₃)₂ and P(O)(OCH₃)OH. In still further embodiments of the invention, R⁸ in the compounds of Formula I is selected from H, C(O)CH₃, and P(O)(OH)₂.

In the method of the invention, R⁹ in the compounds of Formula I is selected from H, F, N₃, CN, C₁-C₆alkyl. Suitably R⁹ is H.

In the method of the invention, X—Y in the compounds of Formula I is selected from —CH₂—O—, —O—CH₂— and —S—CH₂—. Suitably X—Y is —O—CH₂—.

It is an embodiment of the invention that R³ is OH and Z is Formula II. In these compounds the keto tautomeric form is preferred. Accordingly, it is an embodiment of the invention that the compound of Formula I in the method of the invention has the following structure.

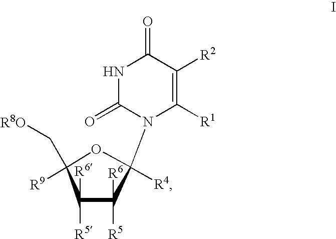

In specific embodiments of the invention, the compound of Formula I in the method of the invention for treating cancer is selected from:
5-fluoro-6-azido-uridine;
5-fluoro-6-azido-uridine-5′-O-monophosphate;
5-fluoro-6-amino-uridine;
5-fluoro-6-amino-uridine-5′-O-monophosphate;
5-fluoro-6-azido uridine 5′-acetate;
5-fluoro-6-azido 2′-deoxyuridine;
5-fluoro-6-azido-2′-deoxyuridine-5′-O-monophosphate;
5-fluoro-6-amino-uridine 5′-acetate;
5-fluoro-6-amino-2′-deoxyuridine;
5-fluoro-6-amino-2′-deoxyuridine-5′-O-monophosphate;
6-iodo-uridine;
6-iodo-uridine-5′-O-monophosphate;
6-iodo-uridine 5′-acetate;
6-iodo-2′-deoxyuridine;
6-iodo-2′-deoxyuridine-5′-O-monophosphate;
6-methyl-uridine;
6-methyl-uridine-5′-O-monophosphate;
6-methyl-uridine 5′-acetate;
6-methyl-2′-deoxyuridine;
6-methyl-2′-deoxyuridine-5′-O-monophosphate;
6-hydoxyamino-uridine;
6-hydroxyamino-uridine-5′-O-monophosphate;
6-hydroxyamino-uridine 5′-acetate;
6-hydroxyamino-2′deoxyuridine;
6-hydroxyamino-2′deoxyuridine-5′-O-monophosphate;
6-formyl-uridine;
6-formyl-uridine-5′-O-monophosphate;
6-formyl-uridine 5′-acetate;
6-formyl-2′deoxyuridine;
6-formyl-2′deoxyuridine-5′-O-monophosphate;
5-fluoro-6-formyl-uridine;
5-fluoro-6-formyl-uridine-5′-O-monophosphate;
5-fluoro-6-formyl-uridine 5′-acetate;
5-fluoro-6-formyl-2′deoxyuridine;
5-fluoro-6-formyl-2′deoxyuridine-5′-O-monophosphate;
5-fluoro-6-ethyl-uridine;
5-fluoro-6-ethyl-uridine-5′-O-monophosphate;
5-fluoro-6-ethyl-uridine 5′-acetate;
5-fluoro-6-ethyl-2′deoxyuridine;
5-fluoro-6-ethyl-2′deoxyuridine-5′-O-monophosphate,
   and tautomers thereof and pharmaceutically acceptable salts, solvates, and prodrugs thereof.

In other embodiments of the invention, the compound of Formula I in the method of the invention for the treatment cancer is selected from:
5-fluoro-6-azido-uridine;
5-fluoro-6-azido-uridine-5′-O-monophosphate;
5-fluoro-6-amino-uridine;
5-fluoro-6-amino-uridine-5′-O-monophosphate;
5-fluoro-6-azido uridine 5′-acetate;
5-fluoro-6-azido 2′-deoxyuridine;
5-fluoro-6-azido 2′-deoxyuridine-5′-O-monophosphate;
5-fluoro-6-amino uridine 5′-acetate;
5-fluoro-6-amino 2′-deoxyuridine;
5-fluoro-6-amino 2′-deoxyuridine-5′-O-monophosphate;
5-fluoro-6-formyl-uridine;
5-fluoro-6-formyl-uridine-5′-O-monophosphate;
5-fluoro-6-formyl-uridine 5′-acetate;
5-fluoro-6-formyl-2′deoxyuridine;
5-fluoro-6-formyl-2′deoxyuridine-5′-O-monophosphate;
5-fluoro-6-ethyl-uridine;
5-fluoro-6-ethyl-uridine-5′-O-monophosphate;
5-fluoro-6-ethyl-uridine 5′-acetate;
5-fluoro-6-ethyl-2′deoxyuridine;
5-fluoro-6-ethyl-2′deoxyuridine-5′-O-monophosphate, and
   tautomers thereof and pharmaceutically acceptable salts, solvates, and prodrugs thereof.

In other embodiments of the invention, the compound of Formula I in the method of the invention for the treatment cancer is selected from:
6-iodo-uridine;
6-iodo-uridine-5′-O-monophosphate;
6-iodo-uridine 5′-acetate;
6-iodo-2′-deoxyuridine;
6-iodo-2′-deoxyuridine-5′-O-monophosphate, and
   tautomers thereof and pharmaceutically acceptable salts, solvates, and prodrugs thereof.

All of the compounds of Formula I have more than one asymmetric centre. Where the compounds according to the invention possess more than one asymmetric centre, they may exist as diastereomers. It is to be understood that all such isomers and mixtures thereof in any proportion are encompassed within the scope of the present invention. In suitable embodiments of the invention, the stereochemistry is that found in the natural form of uridine as depicted above. It is to be understood that while, the relative stereochemistry of the compounds of Formula I is suitably as shown above, such compounds of Formula I may also contain certain amounts (e.g. less than 20%, preferably less than 10%, more preferably less than 5%) of compounds of Formula I having alternate stereochemistry.

In further embodiments, the present invention includes a use of a compound selected from a compound of Formula I as defined above, tautomers thereof, and pharmaceutically acceptable salts, solvates, and prodrugs thereof, for the treatment of cancer as well as a use of a compound selected from a compound of Formula I as defined above, tautomers thereof, and pharmaceutically acceptable salts, solvates, and prodrugs thereof, for the preparation of a medicament for treatment of cancer.

Also including in the present invention is a compound selected from a compound of Formula I, tautomers thereof, and pharmaceutically acceptable salts, solvates, and prodrugs thereof, for use in treating cancer.

According to another aspect of the present invention, there is included a pharmaceutical composition for the treatment of cancer comprising an anticancer effective amount of a compound selected from a compound of Formula I as defined above, and pharmaceutically acceptable salts, solvates, and prodrugs thereof, and a pharmaceutically acceptable carrier or diluent.

The compounds of the invention are suitably formulated into pharmaceutical compositions for administration to human subjects in a biologically compatible form suitable for administration in vivo.

The compositions containing the compounds of the invention can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of Formula I may be used pharmaceutically in the form of the free base, in the form of salts, solvates and as hydrates. All forms are within the scope of the invention. Acid and basic addition salts may be formed with the compounds of the invention for use as sources of the free base form even if the particular salt per se is desired only as an intermediate product as, for example, when the salt is formed only for the purposes of purification and identification. All salts that can be formed with the compounds of the invention are therefore within the scope of the present invention.

In accordance with the methods of the invention, the described compounds of the invention, may be administered to a patient in a variety of forms depending on the selected route of administration, as will be understood by those skilled in the art. The compounds of the invention may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound of the invention may also be administered parenterally. Solutions of a compound of the invention can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compounds of the invention, may be administered to an animal, and most relevantly to a human patient alone or in combination with pharmaceutically acceptable carriers, as noted above, the proportion of which is determined by the solubility and chemical nature of the compound, chosen route of administration and standard pharmaceutical practice.

The compounds of the invention, can be used alone or contemporaneously with other agents that inhibit ODCase activity, or inhibit ODCase activity and other targets, or in combination with other types of treatment (which may or may not modulate ODCase) for treating cancer.

### III. Methods of Preparing Compounds of the Invention

In accordance with another aspect of the present invention, the compounds of the invention can be prepared by processes analogous to those established in the art. In particular, reactions for functionalizing the 5 and/or 6 position of a uracil, cytosine or thymine ring are well known. For example, treatment of uracil, cytosine or thymine with a strong base, such as an alkyl lithium or lithium diisopropyl amide, at reduced temperatures, such at about −60° C. to about −90° C., followed by reaction with a reagent of the Formula R¹-LG, where R¹ is as defined in Formula I and LG is a suitable leaving group, such as halo, provides a compound substituted at the 6-position of the pyrimidine ring with R¹. Compounds substituted with a suitable leaving group, such as I or Br, at the 5-position of the pyrimidine ring of uracil or cytosine are commercially available or are known in the art. These compounds may be converted to their corresponding anions at reduced temperatures, such at about −60° C. to about −90° C., and reacted with a reagent of the Formula R²-LG, wherein R² is as defined in Formula I and LG is a suitable leaving group, such as halo to provide a compound substituted at the 5-position of the pyrimidine ring with R². Conversion of various R¹ groups into other R¹ groups can be done using standard chemistries known to those skilled in the art. For example, azido groups may be reduced to provide amino groups which many be monoalkylated, dialkylated or acylated using known methods.

Pyrimidine compounds may be reacted with a reagent of the formula Z-LG, wherein Z is as defined in Formula I and LG is a suitable leaving group, under standard conditions to provide nucleosides of Formula I or precursors to Formula I. Such reactions would be well known to those skilled in the art. Substitution of the appropriate R¹, R² and/or R³ groups on the pyrimidine ring may be done before or after the coupling of the pyrimidine ring with Z.

Pyrimidine compounds and reagents of the Formula Z-LG are commercially available or may be prepared using methods known in the art. Acylation or addition of the phosphate group on to the 5′ position of the nucleoside may be performed using known reactions.

The incorporation of monophosphate groups at the R⁸ position of the compounds of Formula I can be done using methods known in the art, for example, by reacting the free hydroxy compound with phosphorus oxychloride in the presence of base, such as an organic amine, for example pyridine. The resulting compounds may be converted to their basic salts by neutralization with a suitable base, for example, ammonium hydroxide.

Uracil compounds may be converted to their corresponding cytosine derivatives, for example, by reaction with diphenylphosphorochloridate and 3-nitrotriazole, followed by treatment with ammonia in methanol using methods known in the art.

In some cases the chemistries outlined above may have to be modified, for instance by use of protective groups, to prevent side reactions due to reactive groups, such as reactive groups attached as substituents. This may be achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the invention will vary depending on the compound and the solvate. In general, solvates are formed by dissolving the compound in the appropriate solvent and isolating the solvate by cooling or using an antisolvent. The solvate is typically dried or azeotroped under ambient conditions.

Prodrugs of the compounds of Formula I may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

The present invention includes radiolabeled forms of the compounds of the invention, for example, compounds of the invention labeled by incorporation within the structure of ³H, ¹¹C or ¹⁴C or a radioactive halogen such as ¹²⁵I and ¹⁸F. A radiolabeled compound of the invention may be prepared using standard methods known in the art. For example, tritium may be incorporated into a compound of the invention using standard techniques, for example by hydrogenation of a suitable precursor to a compound of the invention using tritium gas and a catalyst. Alternatively, a compound of the invention containing radioactive iodine may be prepared from the corresponding trialkyltin (suitably trimethyltin) derivative using standard iodination conditions, such as [¹²⁵I] sodium iodide in the presence of chloramine-T in a suitable solvent, such as dimethylformamide. The trialkyltin compound may be prepared from the corresponding non-radioactive halo-, suitably iodo-, compound using standard palladium-catalyzed stannylation conditions, for example hexamethylditin in the presence of tetrakis(triphenylphosphine) palladium (0) in an inert solvent, such as dioxane, and at elevated temperatures, suitably 50-100° C. Further, a compound of the invention containing a radioactive fluorine may be prepared, for example, by reaction of K[¹⁸F]/K222 with a suitable precursor compound, such as a compound of Formula I comprising a suitable leaving group, for example a tosyl group, that may be displaced with the ¹⁸F anion.

### IV. Novel Compounds of the Invention

Novel compounds showing anti-cancer activity are also within the scope of the present invention. Accordingly, the present invention includes all uses of these novel compounds including their use in therapeutic methods and compositions for treating or preventing cancer, their use in diagnostic assays and their use as research tools and as starting materials and/or intermediates in the preparation of other chemical entities.

Accordingly, the present invention provides a compound of Formula Ia selected from:

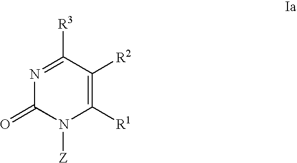

R¹ is selected from CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, NHC(O)OC₁-C₆alkyl and NHC(O)C₁-C₆alkyl;
Z is selected from

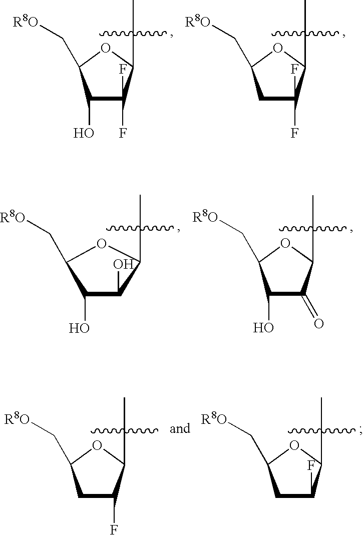

R⁸ is selected from H, C(O)C₁-C₆alkyl, P(O)(OH)₂, P(O)(OC₁-C₆alkyl)₂ and P(O)(OC₁-C₆alkyl)OH,
   tautomers thereof, and pharmaceutically acceptable salts, solvates and prodrugs thereof.

In embodiment of the invention, the compound of Formula Ia is one in which R³ is OH and the compound exists in the following tautomeric form:

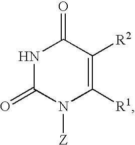

wherein R¹, R² and Z are as defined above.

In another embodiment of the invention, the compound of Formula Ia is one in which R³ is selected from NH₂, NHC(O)OC₁-C₆alkyl and NHC(O)C₁-C₆alkyl, and the compound exists in the following tautomeric form:

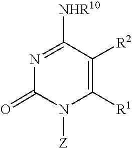

wherein R¹⁰ is selected from H, C(O)OC₁-C₆alkyl and C(O)C₁-C₆alkyl H, R¹, R² and Z are as defined above.

In embodiments of the invention, the compound of Formula Ia is selected from a compound as defined in Table 1, and pharmaceutically acceptable salts, solvates and prodrugs thereof.

The following non-limiting examples are illustrative of the present invention:

### V. Examples

### Example 1

### Synthesis of Compounds Ia and Ib

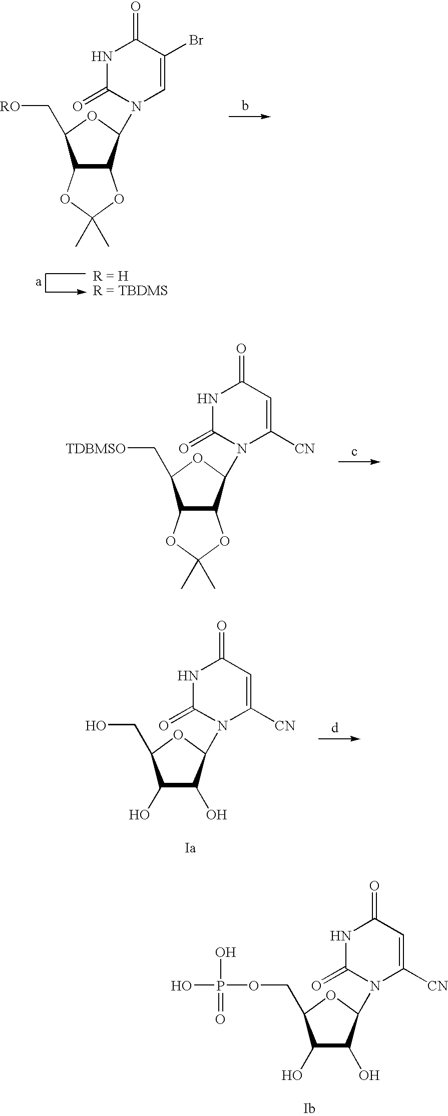

a) TBDMSiCl, imidazole; CH₂Cl₂; b) NaCN DMF; c) 50% aq. TFA; d) POCl₃, Py, H₂O, CH₃CN, 0° C.

Target molecules Ia and Ib were synthesized starting from the 5-bromo-uridine derivative as shown in the above scheme. 2′,3′-O-isopropylidene-5-bromouridine was prepared according to a literature procedure^{xxxiii}. Protection of the primary alcohol in 2′,3′-O-isopropylidene-5-bromouridine as a silyl ether was carried out with t-butyldimethylsilyl chloride (TBDMSiCl) under basic conditions. Fully protected compound was then converted to the 6-cyano derivative using sodium cyanide.^{xxxiv} The deprotection of the protecting groups with 50% aqueous solution of trifluoroacetic acid to yield compound Ia, followed by the mono-phosphorylation with phosphorus oxychloride afforded the desired target molecule Ib.^{xxxv,xxxvi,xxxvii} Finally compound Ib was converted into its ammonium salt using aqueous (NH₄)₂CO₃ solution.

(a) 5′-O-(t-Butyldimethylsilyl)-2′,3′-O-isopropylidene-5-bromouridine: A solution of 2′,3′-O-isopropylidene-5-bromouridine (0.25 g, 0.69 mmol) in dry methylene chloride (CH₂Cl₂) (5.0 mL) was treated with imidazole (Im) (0.093 g, 1.38 mmol) and TBDMSiCl (0.103 g, 0.69 mmol) at 0° C. The reaction mixture was then brought to room temperature, and stirred for 1 h. The reaction mixture was diluted with CH₂Cl₂ and the organic layer was washed with water, brine and dried over Na₂SO₄. Organic layer was concentrated under reduced pressure and the crude compound was purified by silica gel column chromatography (Ethyl Acetate (EtOAc):Hexane, 1:9) to obtain compound 5′-O-(t-Butyldimethylsilyl)-2′,3′-O-isopropylidene-5-bromouridine (0.32 g, 98%) as a foam. ¹H NMR (CDCl₃) δ 0.12 (s, 6H), 0.91 (s, 9H), 1.36 (s, 3H), 1.59 (s, 3H), 3.79 (dd, 1H, J=2.7, 11.5 Hz), 3.93 (dd, 1H, J=2.1, 11.5 Hz), 4.39 (brd, 1H, J=2.1 Hz), 4.67 (dd, 1H, J=3.0, 6.0 Hz), 4.72 (dd, 1H, J=2.1, 6.0 Hz), 5.89 (d, 1H, J=3.0 Hz), 7.90 (s, 1H), 8.41 (brs, 1H).

(b) 5′-O-(t-Butyldimethylsilyl)-2′,3′-O-isopropylidene-6-cyanouridine. A solution of 5′-O-(t-butyldimethylsilyl)-2′,3′-O-isopropylidene-5-bromouridine (0.32 g, 0.71 mmol) in dry DMF (3 mL) was treated with NaCN (0.052 g, 1.07 mmol) at room temperature and the resulting mixture was stirred for 24 h. The reaction mixture was diluted with water (20 mL) and the pH of the solution was brought to ˜6 and was extracted with ethyl acetate (3×20 mL). The combined ethyl acetate layers were washed with brine, dried (Na₂SO₄) and concentrated under reduced pressure. The crude compound was purified by silica gel column chromatography (EtOAc:Hexane, 1:3) to obtain 5′-O-(t-butyldimethylsilyl)-2′,3′-O-isopropylidene-6-cyanouridine in quantitative yield (0.28 g). ¹H NMR (CDCl₃) δ ppm 0.06 (s, 6H), 0.89 (s, 9H), 1.35 (s, 3H), 1.57 (s, 3H), 3.81-3.85 (m, 2H), 4.13-4.18 (m, 1H), 4.76 (dd, 1H, J=4.8, 6.6 Hz), 5.12 (dd, 1H, J=2.4, 6.6 Hz), 6.03 (d, 1H, J=2.4 Hz), 6.29 (s, 1H), 8.88 (brs, 1H); ¹³C NMR (CDCl₃) δ ppm 4.79, 18.84, 25.74, 26.27, 27.52, 63.64, 81.45, 83.65, 88.62, 93.74, 110.84, 113.11, 115.06, 127.71, 148.57, 160.55.

(c) 6-Cyanouridine (Ia). Compound 5′-O-(t-Butyldimethylsilyl-2′,3′-O-isopropylidene-6-cyanouridine (0.12 g, 0.30 mmol) was treated with 50% aqueous trifluoroacetic acid (TFA) (5 mL) at 0° C., then brought to room temperature and stirred for 2 h. Solvent was evaporated and the crude compound was purified by silica gel column chromatography (EtOH: CHCl₃, 1:9) to obtain 6-cyanouridine (Ia) in quantitative yield (0.076 g). UV λₘₐₓ=283 nm; ¹H NMR (dimethylsulfoxide (DMSO)-d₆/D₂O) δ ppm 3.41-3.66 (m, 3H), 4.00 (t, 1H, J=5.7, 6.0 Hz), 4.45 (dd, 1H, J=5.1, 6.0 Hz), 5.73 (d, 1H, J=5.1 Hz), 6.66 (s, 1H).

(d) 6-Cyanouridine-5′-monophosphate (Ib). A stirred solution of POCl₃ (0.3 mL, 3.271 mmol), H₂O (0.037 g, 2.081 mmol) and CH₃CN (2 mL) was treated with pyridine (0.28 mL, 2.081 mmol) at 0° C. and to this, 6-cyanouridine (0.2 g, 0.743 mmol) was added. After 5 h of stirring at 0° C., the reaction mixture was quenched with 50 mL of cold water and the stirring was continued for another 1 h. The reaction mixture was concentrated and the crude compound was purified on Dowex ion-exchange resin (1.0 M formic acid) to obtain 6-cyanouridine-5′-monophosphate (10) (0.12 g, 46%). UV (H₂O): λₘₐₓ=283 nm; ¹H NMR (D₂O) δ 3.98-4.26 (m, 3H), 4.43 (t, 1H, J=6.3 Hz), 4.77 (dd, 1H, J=3.9, 6.3 Hz), 5.95 (d, 1H, J=3.9 Hz), 6.64 (s, 1H).

### Example 2

### Synthesis of Compounds Ic, Id, Ie and If

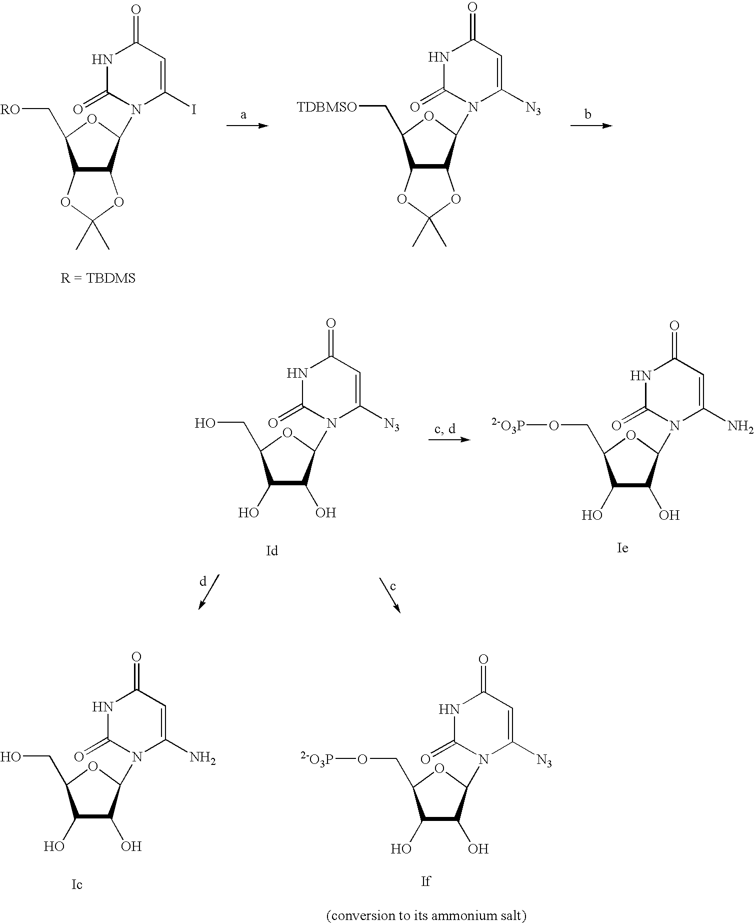

Reaction conditions: (a) NaN₃, DMF, r.t.; (b) 50% TFA, r.t.; (c) POCl₃, pyridine, H₂O, CH₃CN, 0° C.; (d) H₂, PD/C, MeOH, r.t.

Introduction of the iodo moiety at the C-6 position of fully protected uridine was achieved through lithium diisopropyl amide (LDA) and iodine, and further substitution of the iodo by the azido group produced the 6-azido derivative shown in the above scheme.^{xxxviii} Deprotection of the isopropylidene and t-butyldimethylsilyl groups using trifluoroacetic acid yielded 6-azido-uridine Id. Monophosphorylation of Id with phosphorus oxychloride to afford its mononucleotide followed by the reduction of the azido group with Pd/C gave the compound 6-amino-uridine-5′-O-monophosphate Ie in good yield.^{xxxix,xli,xli} Reduction of the azido moiety in compound Id yielded 6-amino-uridine Ic. Phosphorylation of compound Ic with phosphorus oxychloride afforded its mononucleotide 6-azido-uridine-5′-O-monophosphate Ie.

(a) 6-Azido-5′-O-(t-butyldimethylsilyl)-2′,3′-O-isopropylidene uridine. 5′-O-(t-Butyldimethylsilyl)-2′,3′-O-isopropylidene-6-iodo uridine (0.25 g, 0.48 mmol) was dissolved in dry dimethylformamide (DMF) (3 mL) and NaN₃ (0.034 g, 0.53 mmol) was added. The reaction mixture was stirred at room temperature for 1 hr in the dark. Organic solvent was evaporated under vacuum and the crude was dissolved in ethyl acetate (15 mL), washed with brine and dried (Na₂SO₄). Organic layers were evaporated and the crude was purified by silica gel column chromatography (1% ethanol (EtOH):chloroform (CHCl₃)). Purification of the compound and solvent evaporation were performed in the dark to yield the title compound 6-azido-5′-O-(t-butyldimethylsilyl)-2′,3′-O-isopropylidene uridine (0.19 g, 0.44 mmol) in 92% yield as a light brown solid. ¹H NMR (CDCl₃) d 0.06 (s, 6H), 0.89 (s, 9H), 1.34 (s, 3H) 1.54 (s, 3H), 3.74-3.85 (m, 2H), 4.08-4.15 (m, 1H), 4.80 (dd, 1H, J=4.8, 6.3 Hz), 5.14 (dd, 1H, J=1.5, 6.3 Hz), 5.50 (s, 1H), 6.09 (dd, 1H, J=1.5 Hz), 9.12 (brs, 1H).

(b) 6-Azido uridine (Id). A stirred solution of 6-azido-5′-O-(t-butyldimethylsilyl)-2′,3′-O-isopropylidene uridine (0.300 g, 0.683 mmol) was treated with 50% aqueous trifluoroacetic acid (3 mL) at 0° C. The reaction mixture was then brought to r.t. and was stirred for an additional hour. Evaporation of the solvent and purification of the crude by column chromatography (10-15% (ethanol) EtOH in CHCl₃) gave 6-azido uridine Id (0.17 g, 0.61 mmol) in 89% yield as a light brown solid. UV (H₂O): λₘₐₓ=285 nm; ¹H NMR (D₂O) δ 3.77 (dd, 1H, J=5.4, 12.0 Hz), 3.89-4.00 (m, 2H), 4.43 (t, J=6.9 Hz 1H), 4.77 (dd, 1H, J=3.6, 6.9 Hz), 5.76 (s, 1H), 6.07 (d, 1H, J=3.6 Hz). HRMS (ESI) Calculated for C₉H₁₁N₅O₆Na (M+Na⁺) 308.0601, found 308.0597.

(c) 6-Azido uridine-5′-O-monophosphate (If). A stirred solution of water (0.03 g, 1.89 mmol) and POCl₃ (0.28 mL, 2.97 mmol) in anhydrous acetonitrile (3 mL) was treated with pyridine (0.26 mL, 3.24 mmol) at 0° C. and stirred for 10 min. 6-Azido uridine Id was added (0.25 g, 0.68 mmol) and the mixture was stirred for an additional 5 hr at 0° C. The reaction mixture was quenched with 25 mL of cold water and the stirring was continued for another hour. Evaporation of the solvent and purification of the crude by column chromatography (Dowex ion-exchange basic resin, 0.1 M formic acid) gave the mononucleotide If (0.23 g, 0.63 mmol) in 60% yield as syrup. UV (H₂O) λₘₐₓ=283 nm; ¹H NMR (D₂O) δ 3.78-3.85 (m, 1H), 3.89-4.00 (m, 2H), 4.34 (t, J=6.9 Hz 1H), 4.80 (m, 1H), 5.73 (s, 1H), 6.04 (brs, 1H). ³¹P NMR (D₂O) δ ppm 2.47. HRMS (ESI, negative) Calculated for C₉H₁₁N₅O₉P (M⁻) 364.0299, found 364.0307.

(d) 6-Amino uridine-5′-O-monophosphate Ie. The mononucleotide If (0.06 g, 0.15 mmol) was dissolved in 50% aqueous methanol and 10% Pd/C (10 mg) was added. The reaction mixture was stirred for 2 hr under the hydrogen atmosphere at room temperature. The mixture was filtered through Celite® and the solvent was evaporated to dryness to give 6-amino uridine-5′-O-monophosphate Ie as syrup in 85% yield (43 mg, 0.13 mmol). UV (H₂O) λₘₐₓ=270 nm; ¹H NMR (D₂O) δ 3.96-4.05 (m, 2H), 4.12-4.24 (m, 2H), 4.51 (t, J=6.6 Hz 1H), 4.81 (s, 1H), 6.20 (d, J=6.6, 1H). HRMS (ESI, negative) Calculated for C₉H₁₃N₃O₉P (M⁻) 338.0394, found 338.0403.

(e) 6-Amino uridine (Ic). Compound Ic was obtained by treating 6-Azido uridine (Id) with hydrogen in the presence of Pd/C in MeOH using the procedure described above in Example 2(d).

### Example 3

### Synthesis of Compounds Ig and Ih

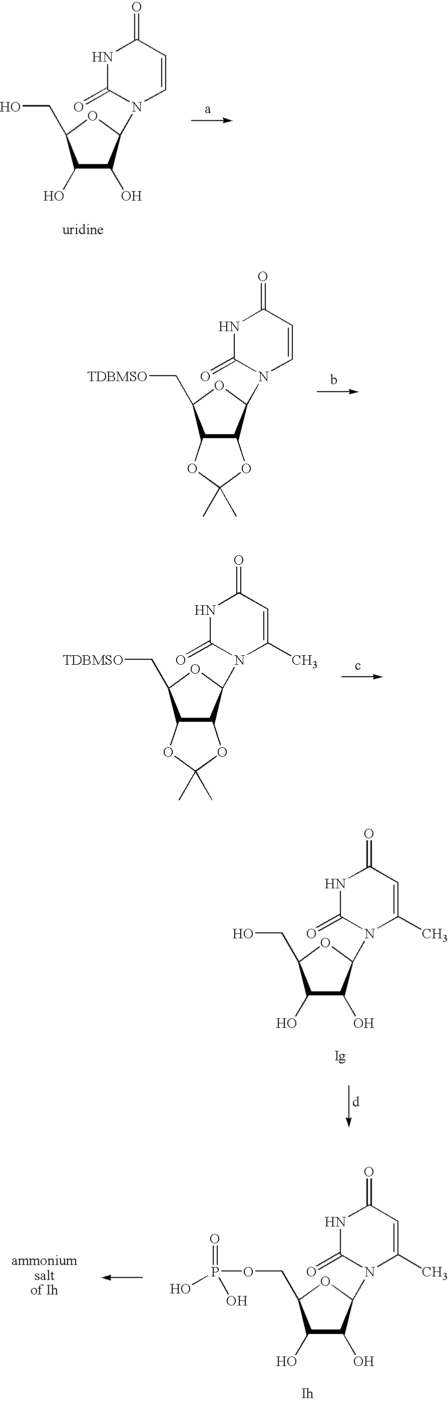

Reaction conditions: (a) i. acetone/H⁺, ii. TBDMSCl, imidazole/CH₂Cl₂, 0-25° C.; (b) LDA, CH₃I, THF, −78° C.; (c) 50% TFA, r.t.; (d) POCl₃, pyridine, H₂O, CH₃CN, 0° C. Target molecules were synthesized from uridine according to literature methods^{xiii}. Introduction of the methyl group in C-6 position was achieved through LDA and methyl iodide.^{xliii} Deprotection of the protecting groups with TFA^{xliv} afforded compund Ig followed by monophosphorylation with phosphorus oxychloride^{xlv,xlvi} afforded the monophosphorylated nucleoside Ih. Finally, monophosphate compound Ih was transformed into the ammonium salt by neutralization with 0.5 M NH₄OH solution at 0° C. and freeze dried to obtain the ammonium salts as powder.

(a) 5′-O-(t-Butyldimethylsilyl)-2′,3′-O-isopropylidene uridine. A stirred suspension of uridine (1 g, 4.098 mmol) in dry acetone (50 mL) was treated with H₂SO₄ (0.5 mL) drop wise at room temperature and the resulting mixture was stirred further 1 h and neutralized with Et₃N. Evaporation of the solvent and purification of the crude by column chromatography (5-8% MeOH in CHCl₃) gave 2′,3′-O-isopropylidene uridine (1.15 g) in quantitative yield as a white solid ¹H NMR (CDCl₃) d: 1.36 (s, 3H, —CH₃), 1.57 (s, 3H, —CH₃), 3.80 (dd, 1H, J=3.3, 12.0 Hz, H-5′), 3.91 (dd, 1H, J=2.7, 12.0 Hz, H-5″), 4.26-4.30 (m, 1H, H-4′), 4.95 (dd, 1H, J=3.3, 6.3 Hz, H-3′), 5.02 (dd, 1H, J=2.7, 6.3 Hz, H-2′) 5.56 (d, 1H, J=2.7 Hz, H-1′), 5.72 (d, 1H, J=8.1 Hz, H-5), 7.36 (d, 1H, J=8.1 Hz, H-6). A stirred solution of 2′,3′-O-isopropylidene uridine (0.2 .g, 0.704 mmol) in dry CH₂Cl₂ (3 mL) was treated with imidazole (0.095 g, 1.408 mmol) and TBDMSCl (0.105 g, 0.704 mmol) at 0° C. The reaction mixture was brought to room temperature and stirred for 1 h. The solvent was evaporated under vacuum and the solid was taken into ethyl acetate (30 mL), washed with water (15 mL), brine (15 mL) and dried (Na₂SO₄). Evaporation of the solvent and purification of crude by column chromatography (5% MeOH in CHCl₃) gave 5′-O-(t-butyldimethylsilyl)-2′,3′-O-isopropylidene uridine (0.268 mg) in 96% yield as a foamy solid. ¹H NMR (CDCl₃): d 0.10 (s, 6H, —CH₃), 0.90 (s, 9H, —CH₃), 1.36 (s, 3H, —CH₃) 1.59 (s, 3H, —CH₃), 3.79 (dd, 1H, J=2.7, 11.2 Hz, H5′), 3.92 (dd, 1H, J=2.4, 11.2 Hz, H-5″), 4.30-4.33 (m, 1H, H-4′), 4.67 (dd, 1H, J=2.7, 6.0 Hz, H-3′), 4.75 (dd, 1H, J=3.0, 6.0 Hz, H-2′), 5.66 (d, 1H, J=8.1 Hz, H-5), 5.96 (dd, 1H, J=3.0 Hz, H-1′), 7.68 (d, 1H, J=8.1 Hz, H-6), 8.47 (brs, 1H, —NH).

(b) 5′-O-(t-Butyldimethylsilyl)-6-methyl-2′,3′-O-isopropylidene uridine. A stirred solution of LDA (0.62 mL, 1.256 mmol, 2.0 M solution in THF) in dry THF (2 mL) was treated with 5′-O-(t-butyldimethylsilyl)-2′,3′-O-isopropylidene uridine (0.25 g, 0.628 mmol) in dry THF 1.5 mL at −78° C. After stirring for 1 h, methyl iodide (0.628 mmol) in dry THF (2 mL) was added and the mixture was stirred for further 5 h at same temperature. The reaction was quenched with acetic acid (AcOH) (0.3 mL), then brought to room temperature and dissolved in ethyl acetate (25 mL). The organic layer was washed with saturated NaHCO₃ solution (10 mL), brine (10 mL) and dried (Na₂SO₄). Evaporation of the solvent and purification of crude by column chromatography (hexanes-ethyl acetate, 70:30) gave 5′-O-(t-butyldimethylsilyl)-6-methyl-2′,3′-O-isopropylidene uridine as a foamy white solid.

(c) 6-Methyl uridine (Ig). A stirred solution of 5′-O-(t-butyldimethylsilyl)-6-methyl-2′,3′-O-isopropylidene (0.300 g) was treated with 50% aqueous trifluoroacetic acid (3 mL) at 0° C. and then brought to room temperature and stirred for 2 h. Evaporation of solvent and purification of crude by column chromatography (10-15% EtOH in CHCl₃) gave 6-methyl uridine (Ig) as a white solid.

(d) 6-Methyl uridine-5′-O-monophosphate (Ih). A stirred solution of H₂O (0.034 g, 1.89 mmol) and POCl₃ (0.277 mL, 2.973 mmol) in dry acetonitrile (3 mL) was treated with pyridine (0.261 mL, 3.24 mmol) at 0° C. and stirred for 10 min. 6-Methyl uridine (Ig) was added (0.675 mmol) and the mixture was stirred for further 5 h at same temperature. The reaction mixture was quenched with 25 mL of cold water and stirring was continued for further 1 h. Evaporation of solvent and purification of crude by column chromatography (Dowex ion-exchange basic resin, 0.1M formic acid) gave 6-methyl uridine-5′-O-monophosphate (Ih) as syrup. The monophosphate derivative was converted to the di-ammonium salt as described earlier.

### Example 4

### Synthesis of 6-iodo uridine (Ii) and 6-iodo-uridine-5′-O-monophosphate (Ij)

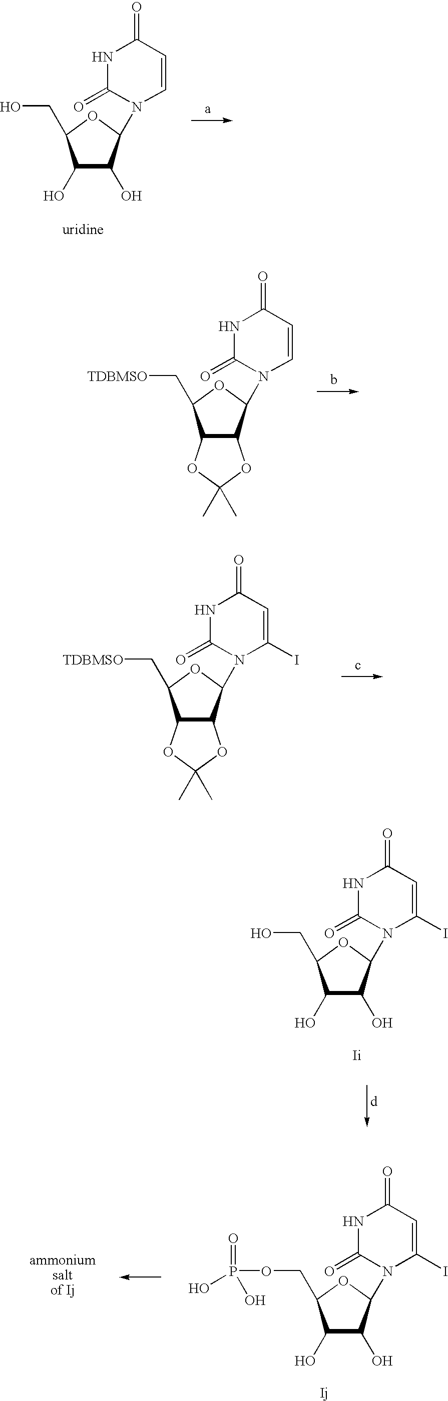

Reaction conditions: (a) i. acetone/H⁺, ii. TBDMSCl, imidazole/CH₂Cl₂, 0-25° C.; (b) LDA, I₂, THF, −78° C.; (c) 50% TFA, r.t.; (d) POCl₃, pyridine, H₂O, CH₃CN, 0° C. Compounds Ii and Ij were synthesized from uridine. Introduction of the iodo moiety at the C-6 position of protected uridine was achieved using lithium diisopropylamide followed by treatment with iodine.^{xlvii} Deprotection with TFA followed gave compound Ii, and the subsequent phosphorylation with phosphorus oxychloride afforded the mononucleotide Ij.^{xlviii,xlix,l} Then, the compound Ij was transformed into its ammonium salt by neutralization with 0.5 M NH₄OH solution at 0° C. and freeze-dried to get the ammonium salt as a powder.

(a) 5′-O-(t-Butyldimethylsilyl)-2′,3′-O-isopropylidene uridine. A stirred suspension of uridine (1 g, 4.1 mmol) in anhydrous acetone (50 mL) was treated with H₂SO₄ (0.5 mL) drop wise at room temperature and the resulting mixture was stirred for an additional hour. The reaction was then neutralized with Et₃N and was concentrated. The crude mixture was purified by column chromatography (5-8% MeOH:CHCl₃) to afford 2′,3′-O-isopropylidene uridine (1.15 g, quant.) as a white solid. ¹H NMR (CDCl₃) d ppm 1.36 (s, 3H, —CH₃), 1.57 (s, 3H, —CH₃), 3.80 (dd, 1H, H-5′), 3.91 (dd, 1H, H-5″), 4.26-4.30 (m, 1H, H-4′), 4.95 (dd, 1H, H-3′), 5.02 (dd, 1H, H-2′) 5.56 (d, 1H, H-1′), 5.72 (d, 1H, H-5),7.36 (d, 1H, H-6).

A stirred solution of 2′,3′-O-isopropylidene uridine (0.2 g, 0.7 mmol) in anhydrous CH₂Cl₂ (3 mL) was treated with imidazole (0.095 g, 1.4 mmol) and TBDMSiCl (0.105 g, 0.7 mmol) at 0° C. The reaction mixture was brought to room temperature and stirred for an additional hour. The solvent was evaporated under vacuum and the crude was dissolved in ethyl acetate (30 mL), washed with water (15 mL), brine (15 mL) and dried (Na₂SO₄). Evaporation of the solvent and purification of the crude by column chromatography (5% MeOH in CHCl₃) yielded 5′-O-(t-butyldimethylsilyl)-2′,3′-O-isopropylidene uridine (0.27 mg, 96% yield) as a foam: ¹H NMR (CDCl₃) d ppm 0.10 (s, 6H, CH₃), 0.90 (s, 9H, CH₃), 1.36 (s, 3H, CH₃) 1.59 (s, 3H, CH₃), 3.79 (dd, 1H, H5′), 3.92 (dd, 1H, H-5″), 4.30-4.33 (m, 1H, H-4′), 4.67 (dd, 1H, H-3′), 4.75 (dd, 1H, H-2′), 5.66 (d, 1H, H-5), 5.96 (dd, 1H, H-1′), 7.68 (d, 1H, H-6), 8.47 (brs, 1H, —NH).

(b) 5′-O-(t-Butyldimethylsilyl)-6-iodo-2′,3′-O-isopropylidene uridine. A stirred solution of LDA (0.62 mL, 1.3 mmol, 2.0 M solution in THF) in anhydrous THF (2 mL) was treated with 5′-O-(t-butyldimethylsilyl)-2′,3′-O-isopropylidene uridine (0.25 g, 0.6 mmol) dissolved in 1.5 mL anhydrous THF, at −78° C. After stirring for 1 h, iodine (0.16 g, 0.6 mmol) in anhydrous THF (2 mL) was added and the mixture was stirred for an additional 5 h at the same temperature. The reaction was quenched with AcOH (0.3 mL), then brought to room temperature and dissolved in ethyl acetate (25 mL). The organic layer was washed with saturated NaHCO₃ solution (10 mL), 5% Na₂S₂O₃ solution (10 mL), brine (10 mL) and dried (Na₂SO₄). Evaporation of the solvent and purification of the crude by column chromatography (hexanes-ethyl acetate, 70:30) gave 5′-O-(t-butyldimethylsilyl)-6-iodo-2′,3′-O-isopropylidene uridine (0.224 g, 68%) as a yellow foam: ¹H NMR (CDCl₃) d ppm 0.06 (s, 6H, CH₃), 0.89 (s, 9H, 3CH₃), 1.35 (s, 3H, CH₃) 1.56 (s, 3H, CH₃), 3.76-3.86 (m, 2H, H5′, H-5″), 4.15-4.20 (m, 1H, H-4′), 4.81 (dd, 1H, J=4.2, 6.3 Hz, H-3′), 5.18 (dd, 1H, J=2.0, 6.3 Hz, H-2′), 6.09 (s, 1H, H-5), 6.45 (dd, 1H, J=2.0 Hz, H-1′), 8.78 (brs, 1H, NH).

(c) 6-Iodo-uridine (Ii). A stirred solution of 5′-O-(t-butyldimethylsilyl)-6-iodo-2′,3′-O-isopropylidene uridine (0.300 g, 0.572 mmol) was treated with 50% aqueous TFA (3 mL) at 0° C., brought to room temperature and stirred for 2 h in the dark. Evaporation of the solvent and purification of the crude by column chromatography (10-15% EtOH in CHCl₃) afforded 6-iodo uridine Ii (0.182 g, 0.49 mmol, 86%) as a light brown solid. UV (H₂O): λₘₐₓ=268 nm (e=8975); ¹H NMR (D₂O) δ ppm 3.77 (dd, 1H, H-5′), 3.91 (dd, 1H, H-5″), 3.978-4.032 (m, 1H, H-4′), 4.43 (t, 1H, H-3′), 4.84 (dd, 1H, H-2′), 6.06 (d, 1H, H-1′), 6.67 (s, 1H, H-5). HRMS (ESI) calculated for C₉H₁₁N₂O₆NaI (M+Na⁺) 392.9554, found 392.9565.

(d) 6-Iodo uridine-5′-O-monophosphate (Ij). A stirred solution of H₂O (0.034 g, 1.89 mmol) and POCl₃ (0.28 mL, 2.97 mmol) in anhydrous acetonitrile (3 mL) was treated with pyridine (0.261 mL, 3.24 mmol) at 0° C. and stirred for 10 min. 6-Iodo uridine (0.250 g, 0.67 mmol) was added and the mixture was stirred for an additional 5 h at 0° C. The reaction mixture was then quenched with 25 mL of cold water and continued stirring for an additional hour. The evaporation of the solvent and purification of the crude by column chromatography (Dowex ion-exchange basic resin, 0.1 M formic acid) afforded 6-iodo uridine-5′-O-monophosphate (Ij) (0.207 g, 68%) as a syrup. UV (H₂O): λₘₐₓ=267 nm (e=2890); ¹H NMR (D₂O) δ ppm 3.78 (dd, 1H, H-5′), 3.91 (dd, 1H, H-5″), 3.98-4.03 (m, 1H, H-4′), 4.43 (t, H-3′), 4.84 (dd, 1H, H-2′), 6.05 (d, 1H, H-1′), 6.67 (s, 1H, H-5). ³¹P NMR (D₂O) δ ppm 2.214. HRMS (ESI, negative) calculated for C₉H₁₁N₂O₉PI (M⁻) 448.9252, found 448.9263.

### Example 5

### Synthesis of Compounds Ik, Ii and Im

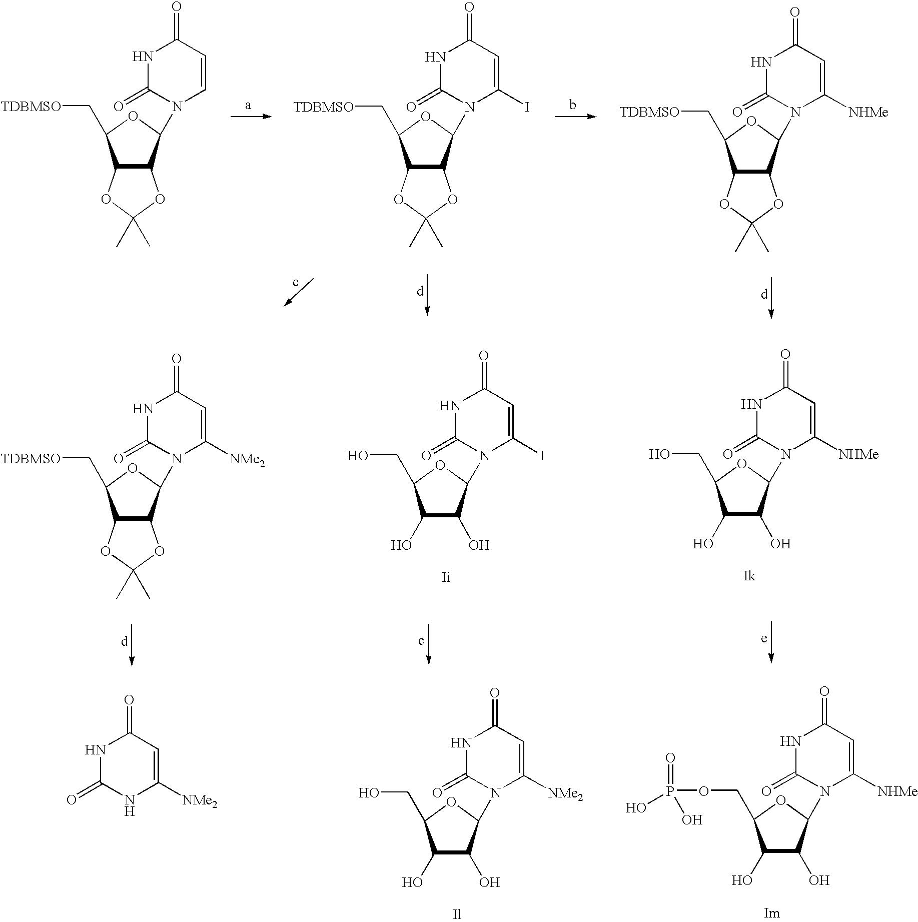

Reaction conditions: (a) THF, I₂, −78° C., LDA; (b) NH₂Me, EtOH, TEA; (c) NHMe₂, EtOH, TEA; (d) TFA, H₂O; (e) POCl₃, pyridine, H₂O, CH₃CN.

(a) 5′-O-(t-Butyldimethylsilyl)-6-N-methylamino-2′,3′-O-isopropylidene uridine. 5′-O-(t-Butyldimethylsilyl)-6-iodo-2′,3′-O-isopropylidene uridine (262 mg, 0.5 mmol) was dissolved 20 mL of dry ethanol, then methylamine (187 mg) was added, followed by adding triethyl amine (1 mL). Reaction mixture was stirred at rt for 3 h and all the start material was consumed. The reaction mixture was evaporated to dryness and purified by column chromatography, (CHCl₃:MeOH=9:1) to obtain 98 mg of 5′-O-(t-Butyldimethylsilyl)-6-methylamino-2′,3′-O-isopropylidene uridine (yield 50%).

(b) 5′-O-(t-Butyldimethylsilyl)-6-N,N-dimethylamino-2′,3′-O-isopropylidene uridine. The procedure was the same as above, except that dimethyl amine was used instead of methyl amine. The reaction was complete in 3 h. The solvent was Hexane: EtoAc=1:1 to purify the product using column chromatography (yield 77.1%).

(c) 6-N,N-Dimethylamino uracil. TFA (10 mL) and H₂O (10 mL) were mixed and cooled to 0° C. and added to the flask with 5′-O-(t-butyldimethylsilyl)-6-N,N-dimethylamino-2′,3′-O-isopropylidene uridine. The mixture was stirred at this temperature for 2 h, followed by an additional hour at room temperature. The mixture was evaporated to dryness, neutralized the mixture with triethyl amine and the resulting mixture was purified by column chromatography (CHCl₃:MeOH=17:3) to obtain 6-N,N-dimethylamino uracil.

(d) 6-Iodouridine (Ii). 5′-O-(t-Butyldimethylsilyl)-6-iodo-2′,3′-O-isopropylidene uridine was treated with trifluoroacetic acid and the product was purified using column chromatography.

(e) 6-N,N-Dimethylamino uridine (Il)). 6-Iodouridine (Ii) was treated with dimethyl amine in ethanol and triethyl amine, as described above. The product was purified by column chromatography (EtOAc:MeOH=8:1) to get 70 mg of 6-N,N-dimethylamino uridine (Il) with an yield of 90.3%.

(f) 6-N-Methylamino uridine (Ik). 5′-O-(t-Butyldimethylsilyl)-6-N-methylamino-2′,3′-O-isopropylidene uridine was treated with trifluoroacetic acid in water to obtain 6-N-methylamino uridine (Ik).

(g) 6-N-Methylamino uridine-5′-O-monophosphate (Im). A stirred solution of POCl₃ (67 mg, 0.44 mmol), H₂O (5 mg) and CH₃CN (0.5 mL) was treated with pyridine (37 mg) at 0° C. 6-N-Methylamino uridine Ik (30 mg, 0.11 mmol) was added and stirred at this temperature for 3 h. The reaction mixture was quenched with 1 mL of cold water and stirred for an additional hour. The mixture was evaporated under reduced pressure and the residue was purified by HPLC to obtain 2 mg of 6-N-methylamino uridine-5′-O-monophosphate (Im).

### Example 6

### Extension to 5-Fluoro Substituted Analogs

5-Fluoro-6-amino uridine (In), 5-fluoro-6-azido uridine (Io), 5-fluoro-6-iodo uridine (Ip), and their mononucleotide forms (Iq, Ir and Is, respectively) were synthesized using the procedures described in Examples 1-5, by substituting uridine with 5-fluoro uridine as the starting material.

Other compounds can be synthesized by utilizing the appropriately protected nucleosides and substituting the C6 substituents as shown in the above examples. Most of the procedures are common in the literature and can be carried out by persons with technical skills in the art.

### Example 7

### Synthesis of Compounds It and Iu

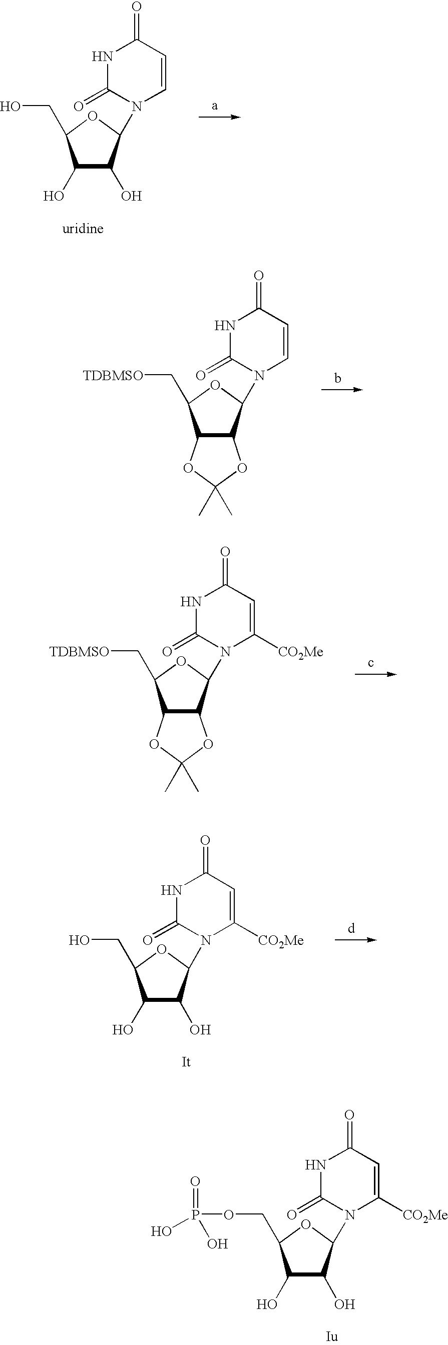

Reaction conditions: (a) i. acetone/H⁺, ii. TBDMSCl, imidazole/CH₂Cl₂, 0-25° C.; (b) LDA, ClCO₂Me, THF, −78° C.; (c) 50% TFA, r.t.; (d) POCl₃, pyridine, H₂O, CH₃CN, 0° C.

Compounds It and Iu were synthesized from uridine. Introduction of methoxycarbonyl in C-6 position was achieved through LDA and methyl chloroformate. Deprotection of the protecting groups with TFA followed by the mono-phosphorylation with phosphorus oxychloride afforded the mono-phosporylated nucleoside. Finally, monophosphate compound Iu was transformed into the ammonium salt by neutralization with 0.5 M NH₄OH solution at 0° C. and freeze dried to yield the ammonium salts as powder.

(a) 5′-O-(^{t}-Butyldimethylsilyl)2′,3′-O-isopropylidene uridine. A stirred suspension of uridine (1 g, 4.098 mmol) in dry acetone (50 mL) was treated with H₂SO₄ (0.5 mL) drop wise at room temperature and the resulting mixture was stirred further 1 h and neutralized with Et₃N. Evaporation of the solvent and purification of the crude by column chromatography (5-8% MeOH in CHCl₃) gave 2′,3′-O-isopropylidene uridine (1.15 g) in quantitative yield as a white solid ¹H NMR (CDCl₃) d: 1.36 (s, 3H, —CH₃), 1.57 (s, 3H, —CH₃), 3.80 (dd, 1H, J=3.3, 12.0 Hz, H-5′), 3.91 (dd, 1H, J=2.7, 12.0 Hz, H-5′), 4.26-4.30 (m, 1H, H-4′), 4.95 (dd, 1H, J=3.3, 6.3 Hz, H-3′), 5.02 (dd, 1H, J=2.7, 6.3 Hz, H-2′) 5.56 (d, 1H, J=2.7 Hz, H-1′), 5.72 (d, 1H, J=8.1 Hz H-5), 7.36 (d, 1H, J=8.1 Hz, H-6). A stirred solution of 2,3-O-isopropylidenuridine (0.2 g, 0.704 mmol) in dry CH₂Cl₂ (3 mL) was treated with imidazole (0.095 g, 1.408 mmol) and TBDMSCl (0.105 g, 0.704 mmol) at 0° C. The reaction mixture was brought to room temperature and stirred for 1 h. The solvent was evaporated under vacuum and the solid was taken into ethyl acetate (30 mL), washed with water (15 mL), brine (15 mL) and dried (Na₂SO₄). Evaporation of the solvent and purification of crude by column chromatography (5% MeOH in CHCl₃) gave the title compound (0.268 mg) in 96% yield as a foamy solid. ¹H NMR (CDCl₃): d 0.10 (s, 6H, —CH₃), 0.90 (s, 9H, —CH₃), 1.36 (s, 3H, —CH₃) 1.59 (s, 3H, —CH₃), 3.79 (dd, 1H, J=2.7, 11.2 Hz, H5′), 3.92 (dd, 1H, J=2.4, 11.2 Hz, H-5″), 4.30-4.33 (m, 1H, H-4′), 4.67 (dd, 1H, J=2.7, 6.0 Hz, H-3′), 4.75 (dd, 1H, J=3.0, 6.0 Hz, H-2′), 5.66 (d, 1H, J=8.1 Hz, H-5), 5.96 (dd, 1H, J=3.0 Hz, H1′), 7.68 (d, 1H, J=8.1 Hz, H-6), 8.47 (brs, 1H, —NH).

(b) 5′-O-(^{t}-Butyldimethylsilyl)-6-Methoxycarbonyl-2′,3′-O-isopropylidene uridine. A stirred solution of 5′-O-(^{t}-butyldimethylsilyl)2′,3′-O-isopropylidene uridine (0.25 g, 0.628 mmol) in dry THF (2 mL) was treated with LDA (0.62 mL, 1.256 mmol, 2.0 M solution in THF) at −78° C. After stirring for 1 h, methylchloroformate (0.048 g, 0.628 mmol) in dry THF (2 mL) was added and the mixture was stirred for further 5 h at same temperature. The reaction was quenched with AcOH (0.3 mL), then brought to room temperature and dissolved in ethyl acetate (25 mL). The organic layer was washed with saturated NaHCO₃ solution (10 mL), 5% Na₂S₂O₃ solution (10 mL), brine (10 mL) and dried (Na₂SO₄). Evaporation of the solvent and purification of crude by column chromatography (hexanes-ethyl acetate, 70:30) gave the title compound (0.18 g) in 64% yield as a syrup. ¹H NMR (CDCl₃): d 0.056 (s, 6H, —CH₃), 0.88 (s, 9H, —CH₃), 1.34 (s, 3H, —CH₃) 1.54 (s, 3H, —CH₃), 3.75 (dd, 1H, J=7.2, 10.9 Hz, H5′), 3.81 (dd, 1H, J=5.1, 10.9 Hz, H5′), 3.93 (s, 3H—CH₃), 4.06-4.12 (m, 1H, H-4′), 4.71 (dd, 1H, J=4.8, 6.4 Hz, H-3′), 5.15 (dd, 1H, J=2.0, 16.4 Hz, H-2′), 5.89 (d, 1H, J=2.1 Hz, H-1′), 6.07 (s, 1H, H-5), 9.32 (brs, 1H, —NH).

(c) 6-Methoxycarbonyl uridine (It). A stirred solution of compound 5′-O-(^{t}-butyldimethylsilyl)-6-methoxycarbonyl-2′,3′-O-isopropylidene uridine (0.23 g, 0.504 mmol) was treated with 50% aqueous TFA (3 mL) at 0° C. and then brought to room temperature and stirred for 2 h. Evaporation of solvent and purification of crude by column chromatography (10-15% EtOH in CHCl₃) yielded It (0.135 g) in 89% yield as a solid. ¹H NMR (DMSO-D₂O): δ 3.37 (dd, 1H, J=6.6, 12.0 Hz, H-5′), 3.54 (dd, 1H, J=3.6, 12.0 Hz, H-5″), 3.62-3.67 (m, 1H, H-4′), 3.80 (s, 3H, —CO₂CH₃), 388-3.97 (m, 1H, H-3′), 4.41 (dd, 1H, J=4.2, 6.3 Hz, H-2′), 5.34 (d, 1H, J=4.2 Hz, H-1′), 5.95 (s, 1H, H-5).

(d) 6-Methoxycarbonyluridine-5′-O-monophosphate (Iu). A stirred solution of H₂O (0.02 g, 1.112 mmol) and POCl₃ (0.16 mL, 1.748 mmol) in dry acetonitrile (3 mL) was treated with pyridine (0.154 mL, 1.907 mmol) at 0° C. and stirred for 10 min. Compound It was added (0.12 g, 0.397 mmol) and the mixture was stirred for further 5 h at same temperature. The reaction mixture was quenched with 25 mL of cold water and stirring was continued for further 1 h. Evaporation of solvent and purification of crude by column chromatography (Dowex ion-exchange basic resin, 0.1M formic acid) gave Iu as syrup. UV (H₂O): λₘₐₓ=274 nm; ¹H NMR (D₂O): δ 3.99 (s, 3H—CO₂CH₃), 4.02-4.08 (m, 2H, H-5′,5″), 4.16-4.23 (m, 1H, H-4′), 4.37 (t, J=6.6 Hz 1H, H-3′), 4.75 (dd, 1H, J=3.3, 6.6 Hz, H-2′), 5.70 (d, 1H, J=3.6 Hz, H-1′), 6.26 (s, 1H, H-5).

### Example 8

### Synthesis of Compounds Iv and Iw

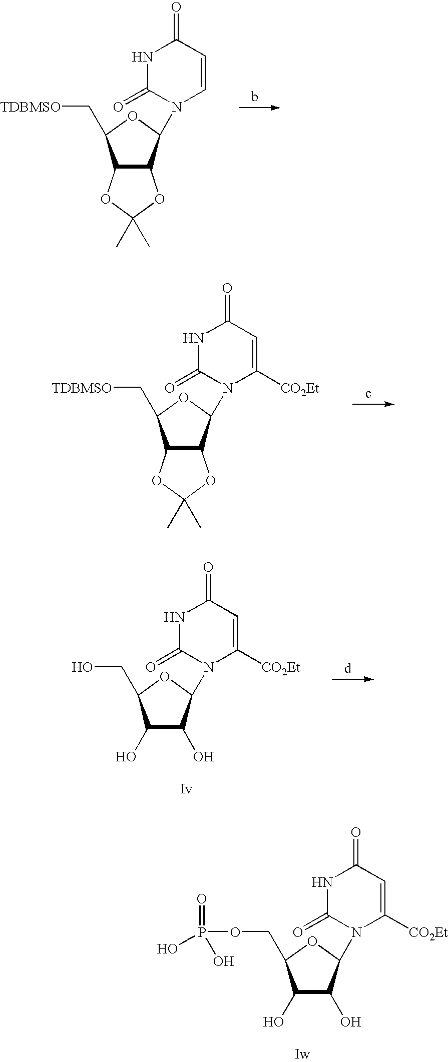

Reaction conditions: (b) LDA, ClCO₂Et, THF, “78° C.; (c) 50% TFA, r.t.; (d) POCl₃, pyridine, H₂O, CH₃CN, 0° C.

Target molecules Iv and Iw were synthesized from 5′-O-(^{t}-butyldimethylsilyl)2′,3′-O-isopropylidene uridine. Introduction of ethoxycarbonyl in C-6 position was achieved through LDA and ethyl chloroformate. Deprotection of the protecting groups with TFA followed by the mono-phosphorylation with phosphorus oxychloride afforded the mono-phosporylated nucleoside Iv. Monophosphate Iw was transformed into the ammonium salt by neutralization with 0.5 M NH₄OH solution at 0° C. and freeze dried to get the ammonium salts as powder.

(a) 5′-O-(-Butyldimethylsilyl)-6-Ethoxycarbonyl-2′,3′-O-isopropylidene uridine. A stirred solution of 5′-O-(^{t}-butyldimethylsilyl)2′,3′-O-isopropylidene uridine (0.25 g, 0.628 mmol) in dry THF (2 mL) was treated with LDA (0.62 mL, 1.256 mmol, 2.0 M solution in THF) at −78° C. After stirring for 1 h, ethyl chloroformate (0.048 g, 0.628 mmol) in dry THF (2 mL) was added and the mixture was stirred for further 5 h at same temperature. The reaction was quenched with AcOH (0.3 mL), then brought to room temperature and dissolved in ethyl acetate (25 mL). The organic layer was washed with saturated NaHCO₃ solution (10 mL), 5% Na₂S₂O₃ solution (10 mL), brine (10 mL) and dried (Na₂SO₄). Evaporation of the solvent and purification of crude by column chromatography (hexanes-ethyl acetate, 70:30) gave the title compound (0.18 g) in 64% yield as a syrup.

(b) 6-Ethoxycarbonyl uridine (Iv). A stirred solution of 5′-O-(^{t}-butyldimethylsilyl)-6-Ethoxycarbonyl-2′,3′-O-isopropylidene uridine (0.23 g, 0.504 mmol) was treated with 50% aqueous TFA (3 mL) at 0° C. and then brought to room temperature and stirred for 2 h. Evaporation of solvent and purification of crude by column chromatography (10-15% EtOH in CHCl₃) gave Iv (0.135 g) in 89% yield as a solid.

(c) 6-Ethoxycarbonyluridine-5′-O-monophosphate (Iw). A stirred solution of H₂O (0.02 g, 1.112 mmol) and POCl₃ (0.16 mL, 1.748 mmol) in dry acetonitrile (3 mL) was treated with pyridine (0.154 mL, 1.907 mmol) at 0° C. and stirred for 10 min. Compound Iv was added (0.12 g, 0.397 mmol) and the mixture was stirred for further 5 h at same temperature. The reaction mixture was quenched with 25 mL of cold water and stirring was continued for further 1 h. Evaporation of solvent and purification of crude by column chromatography (Dowex ion-exchange basic resin, 0.1M formic acid) gave Iw as syrup. Monophosphate Iw was transformed into the ammonium salt by neutralization with 0.5 M NH₄OH solution at 0° C. and freeze dried to get the ammonium salts as powder.

### Example 9

### Synthesis of compound Ix

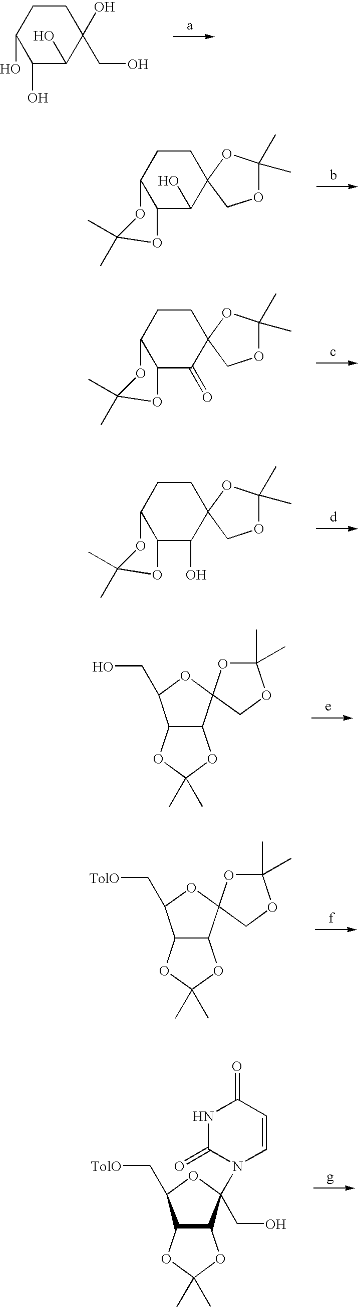

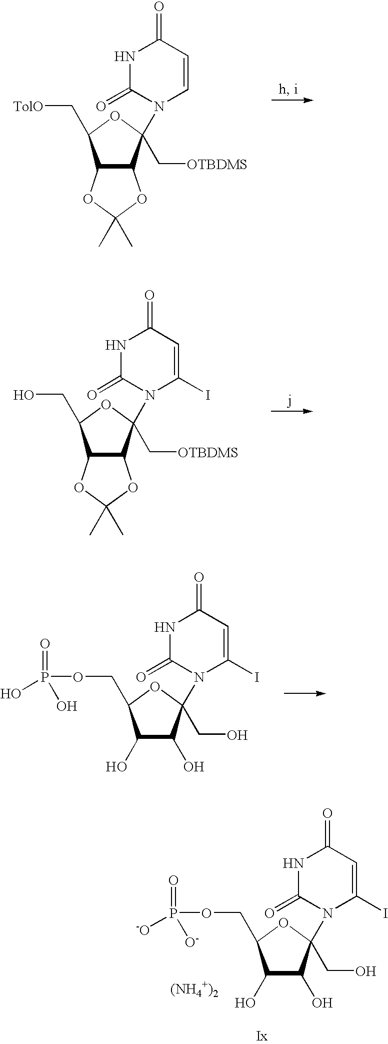

Reaction conditions: (a) acetone, conc. H₂SO₄, r.t.; (b) PCC, 4 angstrom molecular sieves, DCM, r.t.; (c) NaBH₄, EtOH, 0° C.; (d) 50% HClO₄, dimethoxypropoane, acetone, r.t.; (e) p-methyl-benzoyl chloride, triethylamine, DCM; (f) presilylated uracil, TMSOTf, CH₃CN, 0° C. to r.t.; (g) TBDMSCl, DMAP, TEA, DCM, r.t.; (h) LDA, 12, THF, −78° C.; (i) 7N NH₃ in methanol, r.t.; (j) POCl₃, pyridine, H₂O, CH₃CN, 0° C.

D-Fructose was selectively protected, oxidized and then reduced. Rearrangement of the resulting compound was achieved through 2,2-dimethoxypropane and catalytic amount of 50% HClO₄ in acetone. The coupling of the modified sugar with uracil in dry acetonitrile produced a mixture of two isomers, the α-isomer and β-isomer, in ˜1:1 ratio. The desired β-isomer may be iodinated and deprotected with 7N NH₃ in methanol and phosphorylated to give desired monophosphate in one step. The protection groups will come off during hydrolysis. Finally the nucleoside may be converted to ammonium salt Ix.

### (a) 1,2:4,5-Di-O-isopropylidine-β-fructopyranose

To a suspension of D-Fructose (15 g, 83.26 mmol) in dry acetone was added concentrated H₂SO₄ (1.4 mL) by syringe at room temperature. The suspension was stirred at rt and turned clear slowly over a period of 3 h. It was cooled to 0° C. and a solution of NaOH (4.65 g) in water (42 mL) was added to neutralize the sulfuric acid. The solvent was removed under reduced pressure and the residue was extracted with methylene chloride (2×). The combined extracts were washed with water (2×) and then dried over anhydrous Na₂SO₄. After filtration, the solvent was removed to give crude as white solid. The crude was dissolved in dimethyl ether and hexane was added to precipitate the pure product (8.5 g) as a white solid.

### (b) 1,2:4,5-Di-O-isopropylidine-β-D-erytro-2,3-hexodiulo-2,6-pyranose

To a mixture of 1,2:4,5-di-O-isopropylidine-β-fructopyranose (8.24 g, 31.67 mmol, 1.0 equiv.) and powdered 4 Å molecule sieve (20 g) in dichloromethane (200 mL) was added pyridium chlorochromate (PCC) (20.5 g, 3.0 equiv.) in portions over a period of 20 min at room temperature under N₂. The mixture was stirred at r.t. for 5 h and then diluted with large amount of ether and filtered. The filtrate was passed through a pad of celite. The filtrate was passed through a pad of silica gel. The solvent was removed under vacuum to afford product as a white solid (7.8 g, 95 % yield). It was used for next reaction without further purification.

### (c) 1,2:4,5-Di-O-isopropylidine-β-D-psicopyranose

To a solution of 1,2:4,5-di-O-isopropylidine-β-D-erytro-2,3-hexodiulo-2,6-pyranose (16.4 g, 63.52 mmol, 1.0 equiv.) in ethanol (160 mL) was added NaBH₄ solid (1.45 g, 38.11 mmol, 0.6 equiv.) in one portion at 15° C. The mixture was stirred for 1.5 h and then evaporated to almost dryness under reduced pressure. A saturated solution of NH₄Cl (100 mL) was added and the mixture was stirred for 3 h at rt. It was extracted with ether (3×). The combined extracts were washed with brine (2×), dried over anhydrous Na₂SO₄ and filtered. The solvent was removed to give crude as oil, which was used for next reaction directly.

### (d) 1,2:3,4-Di-O-isopropylidine-β-D-psicofuranose

To a solution of crude 1,2:4,5-di-O-isopropylidine-β-D-psicopyranose (14.4 g) in acetone (150 mL) was added dimethoxypropane (4 mL) and 60% HClO₄ (1.0 mL) at 0° C. The mixture was stirred for 3 h at the same temperature. A solution of ammonium hydroxide (2 mL) was added. After evaporation, water was added. The mixture was extracted with ether (3×). The combined extracts were washed with brine (2×), dried over anhydrous Na₂SO₄ and filtered. Evaporation of solvent gave crude product (10.5 g) as an oil, which solidified after vacuum-drying.

### (e) 6-O-(4-Toluoyl)-1,2:3,4-di-O-isopropylidene-D-psicofuranose

To a stirring solution of crude 1,2:3,4-di-O-isopropylidine-β-D-psicofuranose (10.5 g, 40.35 mmol, 1.0 eqiuv.), dimethylaminopyridine (DMAP) (0.49 g, 4.035 mmol, 0.1 eqiuv.) and TEA (20.42 g, 201.75 mmol, 5.0 eqiuv.) was added p-toluoyl chloride (6.86 g, 44.39 mmol, 1.1 eqiuv.) drop wise at 0° C. The resulting light yellow solution was allowed to reach r.t. slowly and then stirred at r.t. overnight. A saturated solution of NaHCO₃ was added. After stirring for 30 min, the organic layer was separated and washed with water (2×) and dried over anhydrous Na₂SO₄. Filtration and evaporation of solvent gave crude, which was purified by column chromatography on silica gel (50:1-20:1 hexane/EtOAc) to provide pure product (10.0 g) as a white solid.

### (f) 1-[3′,4′-O-Isopropylidene-6′-O-(4-toluoyl)-β-D-psicofuranosyl]uracil

To a flask with uracil (2.2 g) were added hexamethyldisilazane (HMDS) (15 mL) and TMSCl (2.4 mL). The resulting suspension was heated to 120° C. under N₂ and stirred at this temperature for 4 h. after which the suspension turned to a clear solution. It was cooled to r.t. and the volatile materials were removed in vacuum. The residue was kept under vacuum for 45 min. and then dissolved in dry acetonitrile (25 mL) and transferred to a solution of 6-O-(4-toluoyl)-1,2:3,4-di-O-isopropylidene-D-psicofuranose (4.2 g, 11.1 mmol, 1.0 eqiuv.) via cannula at rt. The mixture was cooled to 0° C. and TMSOTf (2.96 g, 2.41 mL, 13.31 mmol, 1.2 eqiuv.) was added by syringe. The solution was allowed to reach r.t. slowly and then stirred overnight. A saturated solution of NaHCO₃ was added drop wise. After stirring for 30 min, water was added. The mixture was extracted with EtOAc (3×). The combined extracts were washed with brine (2×), dried over anhydrous MgSO₄ and filtered. Evaporation of solvent gave the cruden product, which was chromatographyed on silica gel (100:1-40:1 CH₂Cl₂/MeOH) to provide pure 1-[3′,4′-O-isopropylidene-6′-O-(4-toluoyl)-α-D-psicofuranosyl]uracil and 1-[3′,4′-O-isopropylidene-6′-O-(4-toluoyl)-β-D-psicofuranosyl]uracil (˜1:1 ratio, 4.26 g, 88.7% yield) as white solids.

### (g) 1-[3′,4′-O-Isopropylidene-6′-O-(4-toluoyl)-β-D-psicofuranosyl]-6-iodouracil (Prophetic)

A stirred solution of 1-[3′,4′-O-isopropylidene-6′-O-(4-toluoyl)-β-D-psicofuranosyl]-uracil (0.628 mmol) in dry tetrahydrofuran (THF) (5 mL) is treated with lithium diisopropyl amide (LDA) (1.984 mmol, 2.0 M solution in THF) in dry THF (2 mL) at −78° C. After stirring for 1 h, iodine (0.161 g, 0.628 mmol) in dry THF (2 mL) is added and the mixture is stirred for further 5 h at same temperature. The reaction is quenched with AcOH (0.3 mL), then brought to room temperature and dissolved in ethyl acetate (25 mL). The organic layer is washed with saturated NaHCO₃ solution (10 mL), brine (10 mL) and dried (Na₂SO₄). Evaporation of the solvent and purification of crude by column chromatography (hexanes-ethyl acetate, 70:30) gives the title compound.

### (h) 1-[1′-O-(t-Butyldimethylsilyl)-3′,4′-O-isopropylidene-6′-O-(4-toluoyl)-β-D-psicofuranosyl]-6-iodouracil (Prophetic)

A mixture of compound 1-[3′,4′-O-isopropylidene-6′-O-(4-toluoyl)-β-D-psicofuranosyl]-6-iodouracil (1.51 g, 3.48 mmol, 1.0 eqiuv.), TBDMSCl (0.63 g, 4.18 mmol, 1.2 eq.), imidazole (1.19 g, 17.42 mmol, and (dimethylamino pyridine) DMAP in dry methylene chloride (50 mL) is stirred at r.t. overnight. A saturated solution of NaHCO₃ is added. The organic layer is separated and the aqueous layer is extracted with methylene chloride. The combined organic layers are washed with water, dried over anhydrous MgSO₄, filtered. The solvent is removed to give crude, which is re-crystallized from ethyl acetate/hexane to afford pure product.

### (i) 1-[1′-O-(t-Butyldimethylsilyl)-3′,4′-O-isopropylidene-β-D-psicofuranosyl]-6-iodouracil (Prophetic)

1-[1′-O-(t-Butyldimethylsilyl)-3′,4′-O-isopropylidene-6′-O-(4-toluoyl)-⊖-D-psicofuranosyl]-6-iodouracil (1.22 g) is dissolved in 7 N NH₃ in methanol. The solution is stirred at r.t. for 2 days. The solvent was removed under reduced pressure. The residue is purified by column chromatography on silica gel (50:1-20:1 CH₂Cl₂/MeOH) to give pure product.

### (j) 1′-Hydroxymethyl-6-iodo-uridine-5′-monophosphate and its Ammonium salt (Ix) (Prophetic)

To a solution of POCl₃ (0.676 g, 4.4 mmol, 4.4 equiv.) in dry acetonitrile (4 mL) is added pyridine (0.380 g, 4.8 mmol, 4.8 equiv.) and water (0.050 g, 2.8 mmol, 2.8 equiv.) at 0° C. After stirring, 1-[1′-O-(t-butyldimethylsilyl)-3′,4′-O-isopropylidene-β-D-psicofuranosyl]-6-iodouracil (0.429 g, 1.0 mmol, 1.0 equiv.) is added. The resulting solution is stirred for 4.5 h and ice-water (20 mL) is added. The mixture is stirred for additional 1.5 h and then evaporated under vacuum. The residue is loaded to a basic resin Dowex column. The column is washed with large amount of water (˜300 mL) and then with 5% formic acid to yield the product acid. This oil is neutralized carefully with ammonium hydroxide 0.5N at 0° C. and freeze dried to give compound Ix.

### Example 10

### Synthesis of Compound Iy

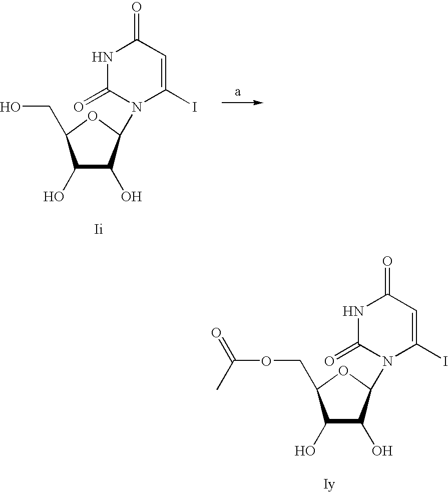

Reaction conditions: (a) acetic anhydride, pyridine, r.t. 1 hour.

A stirred mixture of 6-iodouridine (Ii) in 2 mL of dry pyridine (50 mg, 0.135 mmol) was prepared. To this mixture, 0.068 mmol of acetic anhydride in dry pyridine (2 mL) was added. This mixture was stirred for 25 min, and an additional 0.034 mmol of acetic anhydride in dry pyridine (1 mL) was added, followed by an additional 0.017 mmol of acetic anhydride in pyridine (1 mL). After 15 minutes of stirring, the reaction mixture was evaporated to dryness and the product was purified by column chromatography (3% MeOH in CHCl₃), to obtain 10 mg (18% yield) of the target compound Iy.

### Example 11

### Synthesis of Compounds Iz and Iaa

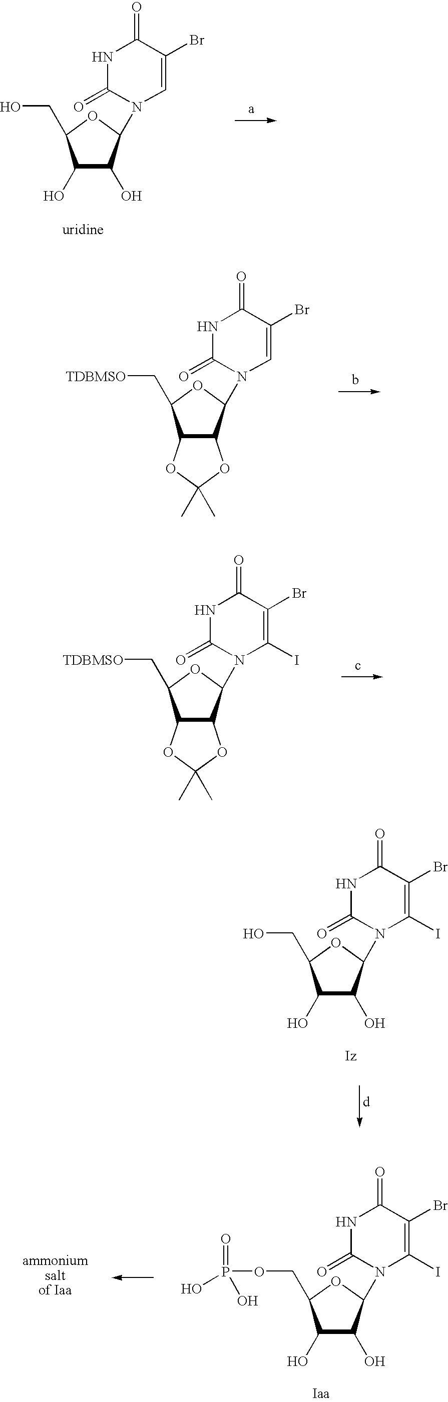

Reaction conditions: (a) i. acetone/H⁺, ii. TBDMSCl, imidazole/CH₂Cl₂, 0-25° C.; (b) LDA, I₂, THF, −78° C.; (c) 50% TFA, r.t.; (d) POCl₃, pyridine, H₂O, CH₃CN, 0° C. Compounds Iz and Iaa were synthesized from 5-bromouridine. Introduction of iodo in C-6 position was achieved through LDA and iodine. Deprotection of the protecting groups with TFA followed by the mono-phosphorylation with phosphorus oxychloride afforded the mono-phosporylated nucleoside. Finally, the monophosphate compound was transformed into the ammonium salt of Iaa by neutralization with a 0.5 M NH₄OH solution at 0° C. and freeze dried to get the ammonium salts as powder.

(a) 5′-O-(^{t}-Butyldimethylsilyl)2′,3′-O-isopropyliden-5-bromo uridine. A stirred suspension of 5-bromouridine (1 g) in dry acetone (50 mL) was treated with H₂SO₄ (0.5 mL) drop wise at room temperature and the resulting mixture was stirred further 1 h and neutralized with Et₃N. Evaporation of the solvent and purification of the crude by column chromatography (5-8% MeOH in CHCl₃) gave 2,3-O-isopropyliden-5-bromouridine (1.15 g) in quantitative yield as a white solid. A stirred solution of 2,3-O-isopropyliden-5-bromouridine (0.2 g) in dry CH₂Cl₂ (3 mL) was treated with imidazole (0.095 g, 1.408 mmol) and TBDMSCl (0.105 g, 0.704 mmol) at 0° C. The reaction mixture was brought to room temperature and stirred for 1 h. The solvent was evaporated under vacuum and the solid was taken into ethyl acetate (30 mL), washed with water (15 mL), brine (15 mL) and dried (Na₂SO₄). Evaporation of the solvent and purification of crude by column chromatography (5% MeOH in CHCl₃) gave the title compound in 96% yield as a foamy solid.

(b) 5′-O-(^{t}-Butyldimethylsilyl)-2′,3′-O-isopropylidene 5-bromo-6-Iodo-uridine. A stirred solution of 5′-O-(^{t}-butyldimethylsilyl)2′,3′-O-isopropyliden-5-bromo uridine (0.628 mmol) in dry THF (5 mL) was treated with LDA (1.984 mmol, 2.0 M solution in THF) in dry THF (2 mL) at −78° C. After stirring for 1 h, iodine (0.161 g, 0.628 mmol) in dry THF (2 mL) was added and the mixture was stirred for further 5 h at same temperature. The reaction was quenched with AcOH (0.3 mL), then brought to room temperature and dissolved in ethyl acetate (25 mL). The organic layer was washed with saturated NaHCO₃ solution (10 mL), brine (10 mL) and dried (Na₂SO₄). Evaporation of the solvent and purification of crude by column chromatography (hexanes-ethyl acetate, 70:30) gave the title compound in 90% yield as a foamy yellow solid.

(c) 5-Bromo-6-Iodo uridine (Iz). A stirred solution of 5′-O-(^{t}-butyldimethylsilyl)-2′,3′-O-isopropylidene 5-bromo-6-Iodo-uridine (0.300 g) was treated with 50% aqueous TFA (3 mL) at 0° C. and then brought to room temperature and stirred for 2 h, light protected. Evaporation of solvent and purification of crude by column chromatography (10-15% EtOH in CHCl₃) gave Iz (0.182 g, 0.492 mmol) in 85% yield as a light brown solid.

(d) 5-Bromo-6-Iodo uridine-5′-O-monophosphate, ammonium salt (Iaa). A stirred solution of H₂O (0.034 g, 1.89 mmol) and POCl₃ (0.277 mL, 2.973 mmol) in dry acetonitrile (3 mL) was treated with pyridine (0.261 mL, 3.24 mmol) at 0° C. and stirred for 10 min. Compound Iz was added (0.675 mmol) and the mixture was stirred for further 5 h at same temperature. The reaction mixture was quenched with 25 mL of cold water and stirring was continued for further 1 h. Evaporation of solvent and purification of crude by column chromatography (Dowex ion-exchange basic resin, 0.1M formic acid) gave the monopshophate derivative in 61% yield as syrup. Finally, monophosphate compound was transformed into the ammonium salt of Iaa by neutralization with 0.5 M NH₄OH solution at 0° C. and freeze dried to get the ammonium salts as powder.

### Example 12

### Compounds Iab-Iah (Table 2)

Further compounds of the invention Iab-Iah are shown in Table 2. The compounds of Table 2 were synthesized using methods analogous to those described in Examples 1-12 in combination with processes known in the art.

### Example 13

### Synthesis of Key Intermediates A, B and C (Prophetic)

The preparation of compounds of Formula I where (i) R⁵ is H, R⁶ is OH, R^{5′} is OH and R^{6′} is H; (ii) R⁵ and R⁶ are together ═O, R^{5′} is OH and R^{6′} is H; (iii) R⁵ and R⁶ are both F, R^{5′} is OH and R^{6′} is H; and (iv) R⁵ and R⁶ are both F and R^{5′} and R^{6′} are both H, and the remaining variables (i.e. R¹-R⁴ and R⁷-R⁹) are as found in the compounds prepared in Examples 1-11, may be prepared from key intermediates A, B, C and D, respectively, which are prepared as described below:

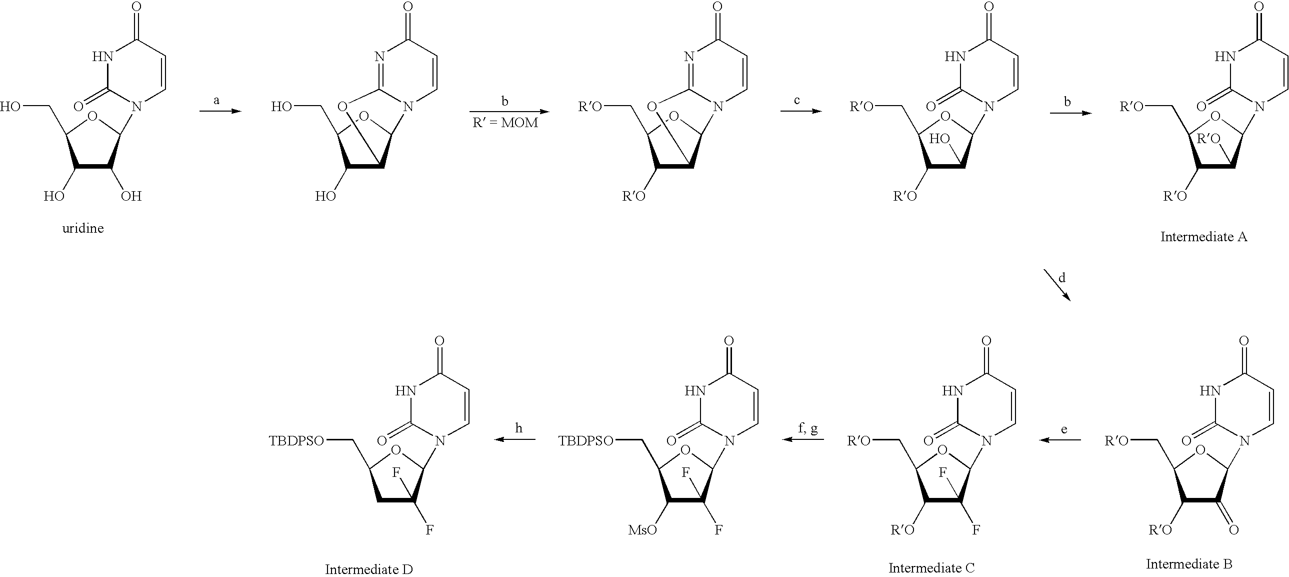

(a) (PhCO)₂O, NaHCO₃, HMPA, 135° C., 40 min; (b) MOMBr, DIPEA, DMF, O° C., 6 h; (c) NaOH, MeOH, 60° C.; (d) Dess-Martin, CH₂Cl₂; (e) DAST CH₂Cl₂, pyridine, —70° C. to reflux; (f) aq. TFA, rt. (g) TBDPSCl, Im, r.t. amd then MsCl, Et₃N; (h) NaH, thioethanol, THF, 0° C. and then Raney Ni, EtOH, reflux.

Using intermediates A, B, C and D and the procedures described in Examples 1-11, the additional representative compounds shown in Table 1 may be prepared. The above examples provide access to other C6-substituted pyrimidine derivatives and someone with knowledge in the art reasonably will be able to design, synthesize and obtain these compounds.

### Example 14

### Anti-Cancer Activity

Molecules containing the core structure, Formula I, with specific substitutions at the C-6 position (R¹) of the pyrimidine moiety are either noncovalent or covalent inhibitors of orotidine monophosphate decarboxylase (ODCase). These molecules may also inhibit other enzymes that accept pyrimidine nucleosides, pyrimidine nucleotides or other appropriately activated forms, as ligands. Examples of such additional enzymes or targets include thymidylate synthase and DNA polymerases etc. All these enzymes are also critical for the synthesis of precursors for, as well as for the synthesis of, DNA and RNA (together termed as nucleic acids). The molecular structures listed above also include, but are not limited to, all chemically-reasonable tautomeric forms of the above structures as well as the prodrugs forms that release the above mentioned compounds and their tautomers. These molecules, described above, exhibit anticancer activities. Such molecules can be used in the treatment of cancer either alone or in combination with other methods of treatment.

(a) In vitro Anticancer Activities:

Thirteen C6 substituted nucleoside derivatives were tested against seventeen cancer cell lines in order to understand their anticancer activity potential. Cell growth was measured in 96 well plates (6 replicates per dose) in which 10⁴ cells were seeded for 3 days with a range of concentrations of compound Ic and Id using the Roche WST-1 assay (Ishiyama M et al (1995) In Vitro Toxicology 8, 187-189) following manufactures directions. This assay relies on the cleavage by cellular enzymes of WST-1 (water soluble tetrazolium: 4-[3-(4-iodophenyl)-2-(4-nitrophenyl)-2H-5-tetrazolio]-1,3-benzene disulfonate to formazan. The amount of conversion is proportional to the mass of living cells and readily read in an ELISA microplate reader using an absorbance wavelength of 450 nm. It has been confirmed using Annexin and Propidium iodine staining that cells exposed to these compounds are indeed dying by apoptosis. IC₅₀ were the mid point in the inhibition curves that were generated.

The results for compounds Ic and Id against various leukemia, lymphoma, and breast cancer cell lines is presented in Table 2. Inhibition constants (IC₅₀s) for these two compounds against various leukemia/lymphoma and breast cancer cell lines (AML1, AML2, SKW3, OCI LY7, OCI MY2, MDA-MB-468 and MCF7) are in the range of 0.7-3.5 mM. In healthy human PBMCs stimulated with concanavalin A and phorbol myristate acetate, inhibitory constants (IC₅₀'s) were greater than 10 mM (Table 2). Based on these promising cell-based results, compounds Ic and Id were considered for in vivo efficacy evaluation.

(b) In vivo Studies:

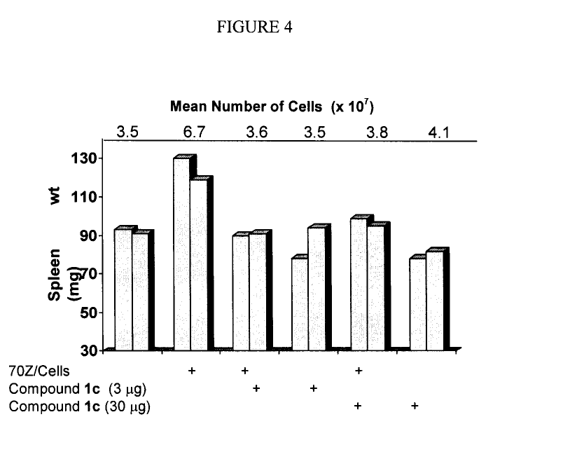

In a pilot study, first the in vivo effects of compound Ic in a murine tumor model (70Z/3) were studied. This murine model served to provide the baseline for toxicity data in a tumor setting. 70Z/3 is a highly pathogenic murine acute lymphoblastic leukemia line which leads to rapid death in mice injected with as few as 100 cell.^{li} In vitro experiments showed that this line, like the human cell lines, was sensitive to compounds Ic in dose ranges similar to the human lines (Table 2). In this pilot study, 10⁶ 70Z/3 cells were injected into syngeneic mice (C57BL6×DBA/2) after which the mice were treated with compound Ic on days 2, 3, 4, 7, 8, and 9 at doses of 0.15 mg/kg or 1.5 mg/kg or with PBS. Some mice were also treated with the compound alone to test for toxic effects in the absence of tumor growth. On day 10, two mice from each group were euthanized and they were examined for the presence of disease. After 70Z/3 cells are injected they home to the spleen and proliferate causing increased spleen weight and cellularity. It was found that the mice which received 70Z/3 cells without treatment had increased spleen weight and cellularity, approximately 30-50% over background (FIG. 4). The spleen weight and cellularity of mice treated with either dose of compound Ic were indistinguishable from the control mice. Injection of Ic alone did not influence spleen weight or cellularity. It would appear from this very small pilot study that mice can tolerate a dose of the drug which is ten times more than a dose which reduces tumor expansion in the spleen (FIG. 4 and Table 2). Two additional mice from each group were followed for an additional 7 days during which mice in the treatment groups were treated daily. During this time both of the untreated mice died (on days 11 and 13) whereas treated mice show no sign of disease progression. NOD-SCID mice were also treated to determine their tolerance for compound Ic at 0.15 mg/kg and 1.5 mg/kg and it was found once again that the mice tolerated these doses without apparent toxicity.

(c) Further In Vitro Anti-Cancer Studies

Cell Lines and Cell Culture Conditions:

A list of cell lines used in this study is presented in Table 4. These cell lines were grown in Iscove's MDM supplemented with 5% fetal calf serum (FCS), 50 μM 2-mercaptoethanol, 100 μg/ml penicillin, and 100 μg/ml streptomycin. Cell lines growing in suspension were maintained at 0.5-1.5×10⁶ cell/ml, while adherent cell lines were maintained at subconfluent densities.

Isolation and Culture of Peripheral Blood Mononuclear Cells:

The protocol for informed consent and the use of human blood was approved by the Ethics Review Committees at the University Health Network in Toronto, Canada. Peripheral blood mononuclear cells (PBMCs) were isolated from peripheral blood from two healthy donors at the Ontario Cancer Institute, University Health Network, by differential density centrifugation over Ficoll-Hypaque (Amersham Biosciences) according to manufacturer's instructions. Briefly, collected blood was diluted in PBS (1:1). A layer of Ficoll-Hypaque was carefully overlayed with diluted blood to a final concentration of 1:1. Cells were spun at 2000 rpm, at room temperature, with no break for 30 minutes. Buffy coat, containing PBMCs, was removed and washed twice in PBS. PBMCs were then stimulated with 1.5 ug/ml of Concanavalin A and seeded to be used in the in vitro cell proliferation assay.

In Vitro Cell Proliferation Assay:

Cell lines were seeded in triplicate at 10⁴ cells/100 μl in 96-well plates. Peripheral blood mononuclear cells were seeded in triplicate at 10⁵ cells/100 μl in 96-well plate. Different test compounds were added immediately at final concentrations ranging between 0 to 100 μM. All compounds were dissolved in sterile deionized water, so water was added to the control wells. On day 3, WST.1 assay (Roche) was used to measure the effect of different test compounds on cell growth. WST.1 assay is a calorimetric assay for the nonradioactive quantification of cell proliferation, cell viability, and cytotoxicity. The assay was performed according to manufacturer's instructions (Roche). It relies on the cleavage by cellular enzymes of WST.1 (water soluble tetrazolium salt: 4-[3-(4-iodophenyl)-2-(4-nitrophenyl)-2H-5-tetrazolio]-1,3-benzene disulfonate) to formazan. The amount of formazan dye formed correlates to the number of metabolically active cells in the culture and is readily read in an ELISA microplate reader using an absorbance wavelength of 440 nm. These OD values are plotted against concentrations of different compounds and IC₅₀ is determined for each compound. Results are presented in Table 5.

Apoptosis Assay:

Cells were seeded in duplicate at 10⁵ cells/ml in 24-well plates. 4 μM final concentration of compounds Io, In or Iag (Table 2) were added immediately after seeding. Water was added to the control wells. Every 24 hr, cells were harvested, washed in PBS, and stained with Annexin V/propidium iodide (PI) (1:200) using Annexin-V-FLUOS staining kit (Roche) according to the manufacturer's instructions. Annexin V is a Ca²⁺⁻ dependent phospholipids-binding protein with high affinity for phosphatidylserine (PS). It can be used as a probe to detect PS exposure on the outer leaflet of the cell membrane and is therefore widely used for detection of apoptotic cells. Unlike apoptotic cells (Annexin V⁺/PI⁻), necrotic cells are Annexin V⁺PI⁺. PI is a DNA stain. FACS was used to analyze the staining. Results are presented in Table 6.

In Vitro Anticancer Activities:

Compounds Io, In, Iag and Iah (Table 2) showed potent anticancer activities against including leukemias (acute myelogenous leukemia-OCI-AML-1, OCI-AML-2, T cell leukemia-SKW3), lymphomas (non-Hodgkin's B cell lymphoma-OCI-Ly-7), multiple myeloma (OCI-My-2). IC₅₀ values for these compounds were in the range of 0.3-2.1 μM (Table 5). Melanoma cell line Lox was highly sensitive to Iag (IC₅₀=0.3 μM). In healthy human PBMCs stimulated with concanavalin A, IC₅₀ for Iab and Iac were greater than 10 μM. Data shown in Table 5 represent averages from at least two experiments.

To determine whether reduced expansion of different cell lines in culture with Io, In, and Iag could be attributed to a decrease in cell survival, we stained treated and control cells with Annexin V and PI. Four cell lines (OCI-AML-1, OCI-Ly7, OCI-My2, and SKW3) were cultured in presence of 4 μM of lab, lac, or lag for 4 days. Cells were harvested daily and Annexin V/PI staining was done by FACS. Our results clearly show an increase in the percentage of apoptotic (annexin V⁺PI⁻) cells cultured with Io, In, or Iag compared to the control cells (Table 6). Increased cell death was first observed on day 2 of culture.

### Example 15

### Enzyme Inhibition Kinetics, with Human ODCase

Reversible Inhibition:

The inhibition of M. thermoautotrophicum ODCase by 5-fluoro-6-amino-UMP (In), 5-fluoro-6-azido-UMP (Io) and 5-fluoro-6-ethyl-UMP (Iah), was evaluated in a competitive inhibition assay. The assay solution containing enzyme (20 nM) and inhibitor (various concentrations) were prepared in 50 mM Tris, 1 mM DTT and transferred to the calorimetric reaction cell. Enzyme activity was measured after a single injection of 5 mM OMP into the 1.3 mL calorimetric cell containing the mixture of enzyme and inhibitor. Final substrate concentration in the reaction cell was 40 μM. The concentration of each inhibitor was varied and the range of the concentrations depended on the initial estimation of the enzyme affinity for a particular inhibitor. The concentration of 5-fluoro-6-amino-UMP was 0, 10, 20, 35, 50 μM while the assay samples with 5-fluoro-6-azido-UMP were prepared with 0, 0.25, 0.50, 0.75, 1.0 μM of inhibitor. The concentration of 5-fluoro-6-ethyl-UMP in the assay samples was 0, 20, 40, 100, and 200 μM.

The inhibition of human ODCase was studied at 37° C. The stock solution of enzyme (60 μM) was prepared in 50 mM Tris (pH 7.5), 20 mM DTT, and 40 mM NaCl and incubated overnight at 4° C. This stock sample was later used to prepare the enzyme assay samples. The enzyme was kept on ice during the experiment. The enzyme stock was diluted with 50 mM Tris buffer containing 1 mM DTT to prepare 60 nM enzyme assay samples. The final substrate concentration was 15 or 20 μM. Concentrated samples of inhibitors were prepared in 50 mM Tris (pH 7.5). The volumes and concentrations of the enzyme and substrate were the same as described above for the reaction without inhibitor. All three compounds were tested in a competitive inhibition assay where enzyme is mixed with the inhibitor and the reaction is initiated by the substrate addition. Assay samples were prepared in 50 mM Tris, 1 mM DTT. The final assay concentration of 5-F-6-azido-UMP was 0, 0.25, 0.50, 0.75, 1.0 μM. The assay samples were prepared with 0, 0.2, 0.4, 1.0, and 2.0 μM and 0, 50, 100, 250, 500 μM 5-fluoro-6-ethyl-UMP. The results are shown in Table 7.

Data Analysis:

The initial data analyses were done in Origin 7.0 software. The raw data representing the heat change over time were converted to the reaction rate at each recorded time point. A Michaelis-Menten plot was constructed and the data were fitted to MM equation (Eq. 1) to determine the kinetic parameters K_{M} and k_{cat}. The molar enthalpy (ΔH) was also automatically calculated.

$$v = \frac{{V}_{max} [ S ]}{{K}_{M} + [ S ]} \qquad ( Equation 1 )$$

To calculate the Kᵢ the data were exported to GraFit 5.0 program. The data points were fitted to the competitive inhibition equation (Eq. 2) and the Kᵢ was estimated from the Dixon plot.

$$v = \frac{{V}_{max} [ S ]}{{K}_{M} ( 1 + \frac{[ I ]}{{K}_{i}} ) + [ S ]} \qquad ( Equation 2 )$$

To determine the inactivation of Hs ODCase following the co-injection of substrate and inhibitor, k_{obs} was first computed from each progress curve. The value (Power, μcal/sec) at each inflection point was normalized by setting the inflection point value to zero. The data representing the change in power over time were fitted to the equation 3 to calculate k_{obs}.

$$[ P ] = \frac{{v}_{i}}{{k}_{obs}} ( 1 - exp ( - {k}_{obs} t ) ) \qquad ( Equation 3 )$$

Where [P] represents the product concentration, vᵢ is the initial reaction rate, and t is time. The calculated k_{obs} and the inhibitor concentrations [I] were used to calculate the inactivation constant K_{I} and the rate of inactivation k_{inact} from the equation:

$${k}_{obs} = \frac{{k}_{inact} [ I ]}{{K}_{I} + [ I ]} \qquad ( Equation 4 )$$

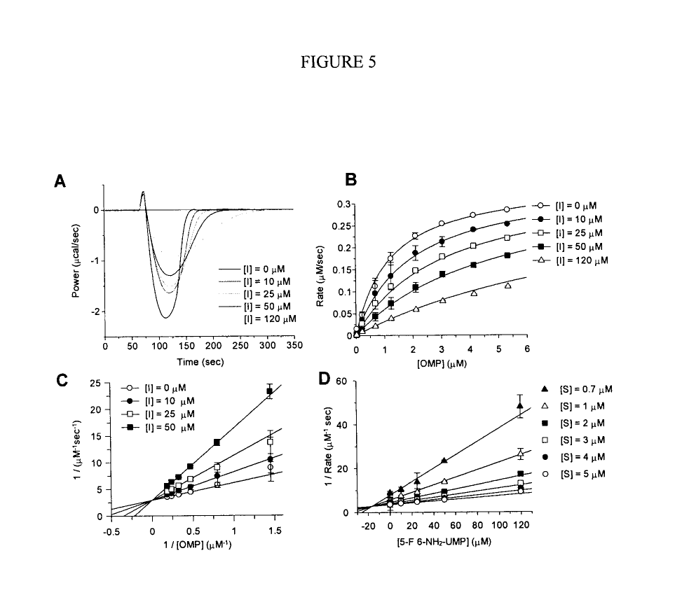

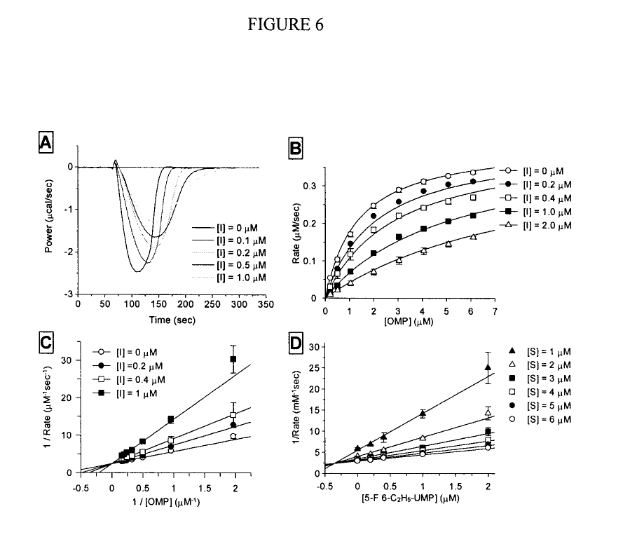

Graphical representations of the results are shown in FIGS. 5-7

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

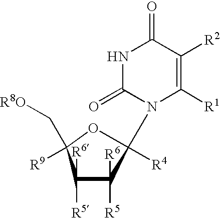

| Cpd # | R¹ | R² | R⁴ | R⁵ | R⁶ | R^{5′} | R^{6′} | R⁸ | R⁹ |
|---|---|---|---|---|---|---|---|---|---|
| Ibb | NH₂ | F | H | H | OH | OH | H | H | H |
| Icc | NH₂ | F | H | H | OH | OH | H | PO₃²⁻ | H |
| Idd | N₃ | F | H | H | OH | OH | H | H | H |
| Iee | N₃ | F | H | H | OH | OH | H | PO₃²⁻ | H |
| Iff | NH₂ | F | H | F | F | OH | H | H | H |
| Igg | NH₂ | F | H | F | F | OH | H | PO₃²⁻ | H |
| Ihh | N₃ | F | H | F | F | OH | H | H | H |
| Iii | N₃ | F | H | F | F | OH | H | PO₃²⁻ | H |
| Ijj | NH₂ | F | H | F | F | H | H | H | H |
| Ikk | NH₂ | F | H | F | F | H | H | PO₃²⁻ | H |
| Ill | N₃ | F | H | F | F | H | H | H | H |
| Imm | N₃ | F | H | F | F | H | H | PO₃²⁻ | H |

| | | | | | | | | |
|---|---|---|---|---|---|---|---|---|
| Inn | NH₂ | F | H | ═O | OH | H | H | H |
| Ioo | NH₂ | F | H | ═O | OH | H | PO₃²⁻ | H |
| Ipp | N₃ | F | H | ═O | OH | H | H | H |
| Iqq | N₃ | F | H | ═O | OH | H | PO₃²⁻ | H |

TABLE 2

| | |
|---|---|
| | I |
| 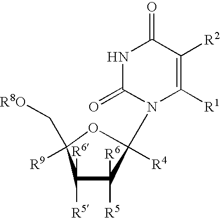 | |

| Cpd # | R¹ | R² | R⁴ | R⁵ | R⁶ | R^{5′} | R^{6′} | R⁸ | R⁹ |
|---|---|---|---|---|---|---|---|---|---|
| Io | N₃ | F | H | OH | H | OH | H | H | H |
| In | NH₂ | F | H | OH | H | OH | H | H | H |
| Iad | CH₃ | H | H | H | H | OH | H | H | H |
| Iae | NHOH | H | H | OH | H | OH | H | H | H |
| Iaf | CHO | H | H | OH | H | OH | H | H | H |
| Iag | CHO | F | H | OH | H | OH | H | H | H |
| Iah | C₂H₅ | F | H | OH | H | OH | H | H | H |

TABLE 3

IC₅₀ (mM)

| | Ic | Id |
|---|---|---|
| CCD967 | >4 | >4 |
| LOX | >4 | 3.8 ± 0.2 |
| SNB19 | >4 | >4 |
| MDA-MB-468 | 3.7 ± 0.1 | 2.5 ± 0.3 |
| PPC1 | >4 | >4 |
| PC3 | >4 | >4 |
| COLO205 | >4 | >4 |
| HS578T | >4 | >4 |
| OCI AML1 | 0.8 ± 0.1 | 0.7 ± 0.1 |
| OCI AML2 | 0.9 ± 0.1 | 0.8 ± 0.2 |
| OCI LY7 | 1.8 ± 0.1 | 1.0 ± 0.2 |
| MCF7 | 1.5 ± 0.1 | 1.0 ± 0.2 |
| OVCAR4 | >4 | >4 |
| T47D | >4 | >4 |
| OCI MY2 | >4 | 3.8 ± 0.0 |
| SKW3 | 0.8 ± 0.1 | 0.7 ± 0.1 |
| 70Z/3 (murine) | 0.3 ± 0.0 | 0.2 ± 0.0 |
| PBMC MN | >10 | >10 |
| PBMC DK | >10 | >10 |

¹Cancer cell lines used in this study: CCD967 - Skin Fibroblast line, LOX - Melanoma, SNB19 - Glioblastoma, PPC1 - Prostate, PC3 - Prostate adenocarcinoma, COLO205 - colorectal adenocarcinoma, OVCAR4 - Ovarian adenocarcinoma, MDA-MB-468 - breast, adenocarcinoma, HS578T - breast, carcinosarcoma, MCF7 - breast, adenocarcinoma, T47D - breast, adenocarcinoma, OCI AML1 - myloid leukemia, OCI AML2 - myloid leukemia, OCI LY7 - B cell lymphoma, OCI MY2 - myeloma, SKW3 - T cell leukemia, 70Z/3 - murine B cell leukemia, PBMC - Peripheral blood mononuclear cells (obtained form two different volunteers, MN and DK).

TABLE 4
(List of cancer cell lines)

| Name | Origin | Source |
|---|---|---|
| OCI-AML-1 | Acute myeloid leukemia | Ontario Cancer Institute |
| OCI-AML-2 | Acute myeloid leukemia | Ontario Cancer Institute |
| OCI-Ly-7 | Diffuse large B cell lymphoma | Ontario Cancer Institute |
| OCI-My-2 | Multiple myeloma | Ontario Cancer Institute |
| SKW-3 | Chronic lymphocytic leukemia-derived T cell line | See Ref. 1. |
| LOX | Melanoma derived from lymph node | NCI Frederick Cancer |
| | | DCT Tumor Repository |
| SNB-19 | Glioblastoma derived from cerebrum | NCI Frederick Cancer |
| | | DCT Tumor Repository |
| OVCAR-4 | Adenocarcinoma derived from ovary | NCI Frederick Cancer |
| | | DCT Tumor Repository |
| COLO-205 | Colorectal adenocarcinoma derived from | ATCC CCL-222 |
| | ascites | |
| PC-3 | Adenocarcinoma derived from prostate | ATCC CRL-1435 |
| T-47D | Ductal carcinoma derived from breast | ATCC HTB-133 |
| MDA-468 | Adenocarcinoma derived from breast | ATCC HTB-132 |
| MCF-7 | Adenocarcinoma derived from breast | ATCC HTB-22 |
| HS-578T | Ductal carcinoma derived from breast | ATCC HTB-126 |
| CCD-967 | Normal skin line | ATCC |

¹T. Hirano, T. Kishimoto, A. Muraguchi, Y. Yamamura, P. Ralph and R. A. Good. In vitro immune response of human peripheral lymphocytes. J. Immunol. 123 (1979) 1133-1140.

TABLE 5
(Anticancer activities expressed as IC₅₀ (μM) against various cell lines)

| | OCI- | OCI- | | | | | | |
|---|---|---|---|---|---|---|---|---|
| | AML- | AML- | OCI- | OCI- | | | | |
| Compd | 1 | 2 | Ly-7 | My-2 | SKW3 | MCF7 | LOX | MDA468 |
| 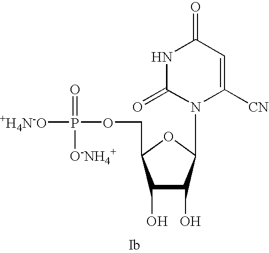 | >50 | >50 | >50 | >50 | >50 | >50 | >50 | >50 |
| 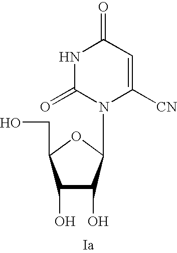 | >50 | >50 | >50 | >50 | >50 | >50 | >50 | >50 |
| 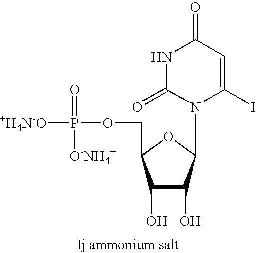 | 35.0 | 22.4 | 46.6 | 26.5 | 8.8 | 43.9 | >50 | >50 |
| 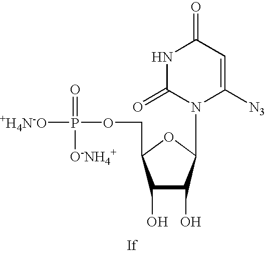 | >50 | >50 | >50 | >50 | >50 | >50 | >50 | >50 |
| 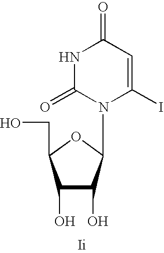 | 15.3 | >50 | >50 | 27.8 | 18.9 | >50 | >50 | >50 |
| 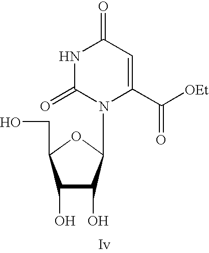 | >50 | >50 | >50 | 42.3 | >50 | >50 | >50 | >50 |
| 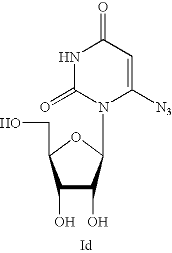 | >50 | >50 | >50 | >50 | >50 | >50 | >50 | >50 |
| 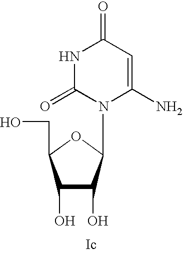 | >50 | >50 | >50 | >50 | >50 | >50 | >50 | >50 |
| 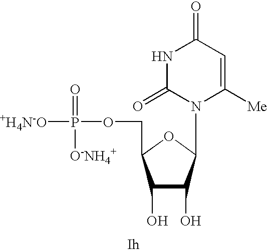 | >50 | >50 | >50 | >50 | >50 | >50 | >50 | >50 |
| 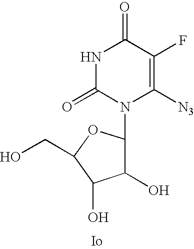 | 0.6 | 0.7 | 0.7 | 0.6 | 0.4 | 1.0 | 2.3 | 1.8 |
| 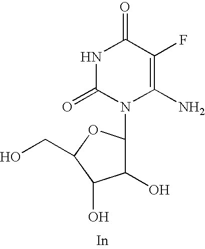 | 0.7 | 0.9 | 1.3 | 0.9 | 0.6 | 1.4 | 3.4 | 2.6 |
| 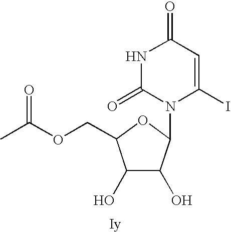 | 15.2 | 11.7 | 36.2 | 1.3 | 1.6 | 4.4 | 1.1 | >50 |
| 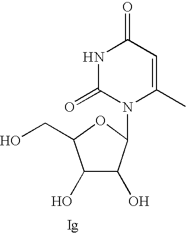 | >50 | >50 | >50 | >50 | >50 | >50 | >50 | >50 |
| 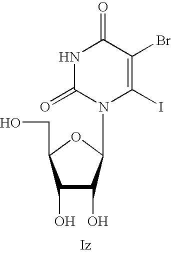 | >50 | >50 | >50 | >50 | >50 | >50 | >50 | >50 |
| 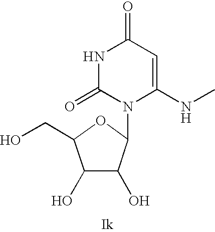 | >50 | >50 | >50 | >50 | >50 | >50 | >50 | >50 |
| 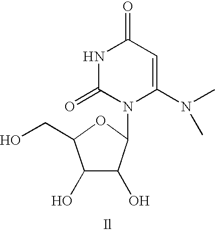 | >50 | >50 | >50 | >50 | >50 | >50 | >50 | >50 |
| 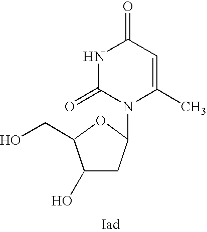 | >50 | >50 | >50 | >50 | >50 | >50 | >50 | >50 |
| 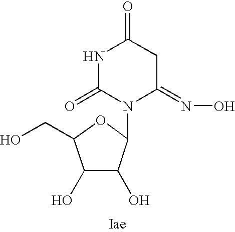 | >50 | >50 | >50 | >50 | >50 | >50 | >50 | >50 |
| 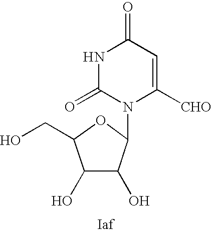 | >50 | >50 | >50 | >50 | >50 | >50 | >50 | >50 |
| 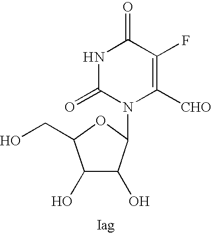 | 0.3 | 0.7 | 0.6 | 0.4 | 0.3 | 23.7 | 0.4 | 1.9 |
| 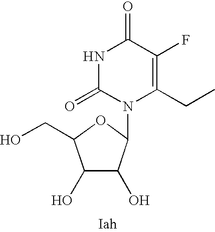 | 2.1 | -ND- | 1.9 | 1.4 | 1.2 | 31.4 | 4.5 | 5.3 |
| 5-Fluoro-uracil | 1.0 | 1.6 | -ND- | -ND- | 2.1 | -ND- | -ND- | -ND- |

| Compd | OCVR4 | COLO205 | CCD967 | HS58T | T47D | PC3 | SNB19 |
|---|---|---|---|---|---|---|---|
| 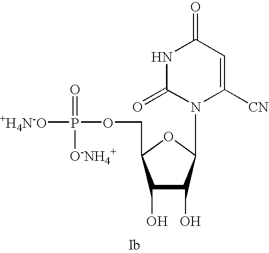 | >50 | >50 | >50 | >50 | >50 | >50 | >50 |
| 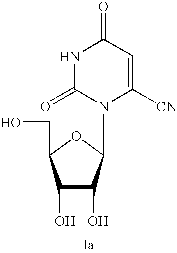 | >50 | >50 | >50 | >50 | >50 | >50 | >50 |
| 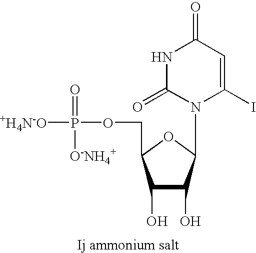 | >50 | >50 | >50 | >50 | >50 | >50 | >50 |
| 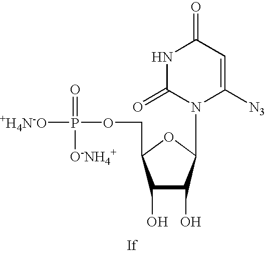 | >50 | >50 | >50 | >50 | >50 | >50 | >50 |
| 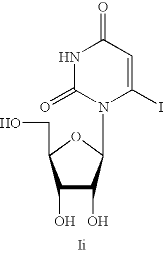 | >50 | >50 | >50 | >50 | >50 | >50 | >50 |
| 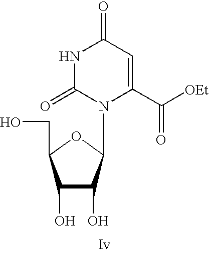 | >50 | >50 | >50 | >50 | >50 | >50 | >50 |
| 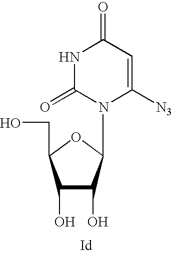 | >50 | >50 | >50 | >50 | >50 | >50 | >50 |
| 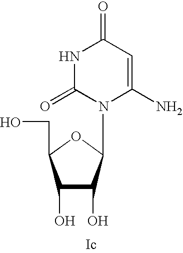 | >50 | >50 | >50 | >50 | >50 | >50 | >50 |
| 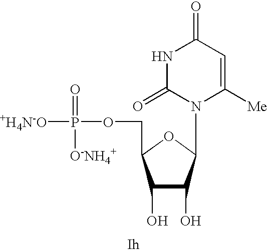 | >50 | >50 | >50 | >50 | >50 | >50 | >50 |
| 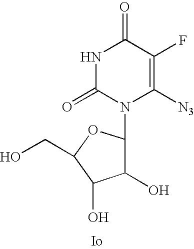 | 1.5 | 3.8 | >4 | >4 | >4 | >4 | >4 |
| 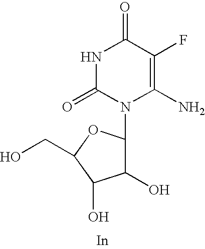 | 2.0 | 3.3 | >4 | >4 | >4 | >4 | >4 |
| 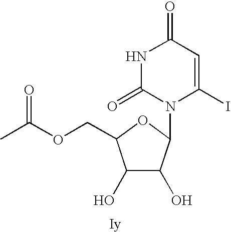 | >50 | >50 | >50 | >50 | >50 | >50 | >50 |
| 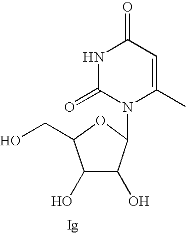 | >50 | >50 | >50 | >50 | >50 | >50 | >50 |
| 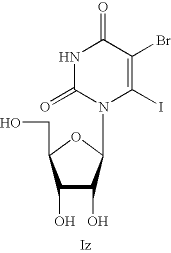 | >50 | >50 | >50 | >50 | >50 | >50 | >50 |
| 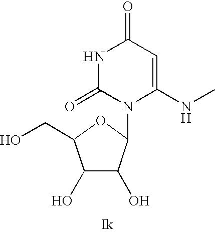 | >50 | >50 | >50 | >50 | >50 | >50 | >50 |
| 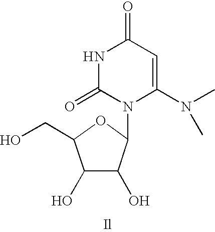 | >50 | >50 | >50 | >50 | >50 | >50 | >50 |
| 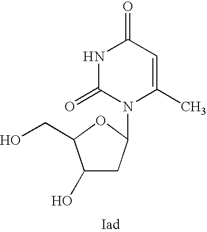 | -ND- | -ND- | -ND- | -ND- | -ND- | -ND- | -ND- |
| 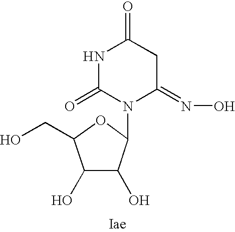 | -ND- | -ND- | -ND- | -ND- | -ND- | -ND- | -ND- |
| 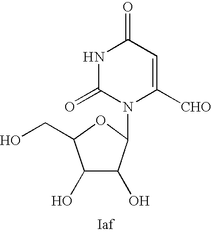 | -ND- | -ND- | -ND- | -ND- | -ND- | -ND- | -ND- |
| 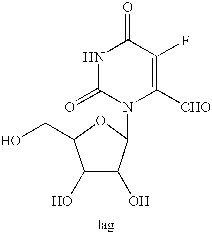 | -ND- | -ND- | -ND- | -ND- | -ND- | >100 | -ND- |
| 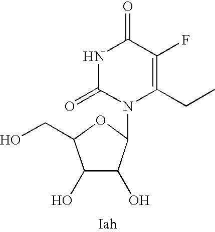 | -ND- | -ND- | -ND- | -ND- | -ND- | >100 | -ND- |
| 5-Fluoro-uracil | -ND- | -ND- | -ND- | -ND- | -ND- | -ND- | -ND- |

TABLE 6
(Induction of apoptosis in cancer cell lines by compounds Io, In and Iag.)
Fold increase in % of apoptotic (annexinV⁺PI⁻) cells:
drug-treated vs.control

| Cell line: | day 1 | day 2 | day 3 |
|---|---|---|---|
| OCI-AML-1 | | | |
| Io | 1.5 | 9.3 | 16 |
| In | 1.3 | 3.4 | 2.3 |
| Iag | 1.4 | 5.3 | 14 |
| SKW3 | | | |
| Io | 1.2 | 4.7 | 4.4 |
| In | 1.0 | 3.9 | 4.9 |
| Iag | 1.2 | 4.6 | 5.2 |
| OCI-My-2 | | | |
| Io | 0.86 | 1.9 | 2.8 |
| In | 0.75 | 1.7 | 2.0 |
| Iag | 0.77 | 1.7 | 2.7 |
| OCI-Ly-7 | | | |
| Io | 0.74 | 1.9 | 6.6 |
| In | 0.62 | 1.8 | 3.9 |
| Iag | 0.82 | 2.1 | 6.2 |

TABLE 7
(Reversible inhibition of ODCases by In, Io and Iah)

| | Hs ODCase | Mt ODCase |
|---|---|---|
| | Kᵢ (μM) | Kᵢ (μM) |
| 5-F-UMP | 97.8 ± 5.9 | 645.7 ± 15 |
| In | 16.6 ± 0.7 | 11.4 ± 0.6 |
| Io | -ND- | 0.357 ± 0.028 |
| (covalent inhibitor) | | |
| Iah | 0.353 ± 0.01 | 29.0 ± 0.3 |

### FULL CITATIONS FOR DOCUMENTS REFERRED TO IN THE SPECIFICATION

^{i.} Warshel, A.; Florian, J. Computer simulations of enzyme catalysis: finding out what has been optimized by evolution. Proc. Natl. Acad. Sci. U.S.A. 1998, 95, 5950-5955.
ⁱⁱ. Miller, B. G.; Wolfenden, R. Catalytic proficiency: The unusual case of OMP decarboxylase. Ann. Rev. Biochem. 2002, 71, 847-885.
ⁱⁱⁱ. Sievers, A.; Wolfenden, R. Equilibrium of formation of the 6-carbanion of UMP, a potential intermediate in the action of OMP decarboxylase. J. Am. Chem. Soc. 2002, 124, 13986-13987.
^{iv}. Snider, M. J.; Wolfenden, R. The rate of spontaneous decarboxylation of amino acids. J. Am. Chem. Soc. 2000, 122, 11507-11508.
^{v}. Reichard, P. The enzymatic synthesis of pyrimidines. Adv. Enzymol. Mol. Biol. 1959, 21, 263-294.
^{vi}. Kotra, L. P.; Pai, E. F.; Bello, A. M.; Fujihashi, M.; Poduch, E. (2005) Inhibitors of orotidine monophosphate decarboxylase (ODCase) activity, U.S. Application No. 60/596,537, patent pending.
^{vii}. Kotra, L. P; Fish, E. N. Novel C-6 substituted pyrimidine nucleosides as antiviral agents. New invention disclosure, University Health Network, September 2006.
^{viii}. Christopherson, R. I.; Lyons, S. D.; Wilson, P. K. Inhibitors of de Novo nucleotide biosynthesis as drugs. Acc. Chem. Res. 2002, 35, 961-971.
^{ix}. Scott, H. V.; Gero, A. M.; O'Sullivan, W. J. In vitro inhibition of Plasmodium falciparum by pyrazofurin, an inhibitor of pyrimidine biosynthesis de novo. Mol. Biochem. Parasitol. 1986, 18, 3-15.
^{x}. Levine, H. L.; Brody, R. S.; Westheimer, F. H. Inhibition of orotidine-5′-phosphate decarboxylase by 1-(5′-phospho-beta-d-ribofuranosyl)barbituric acid, 6 azauridine 5′-phosphate, and uridine 5′-phosphate, Biochemistry 1980, 19, 4993-4999.

^{xi}. Gero, A. M.; O'Sullivan, W. J. Purines and pyrimidines in malarial parasites, Blood Cells 1990, 16, 467-484.
^{xii}. Seymour, K. K.; Lyons, S. D.; Phillips, L.; Rieckmann, K. H.; Christopherson, R. I. Cytotoxic effects of inhibitors of de novo pyrimidine biosynthesis upon Plasmodium falciparum, Biochemistry 1994, 33, 5268-5274.
^{xiii}. Krungkrai, J.; Krungkrai, S. R.; Phakanont, K. Antimalarial activity of orotate analogs that inhibit dihydroorotase and dihydroorotate dehydrogenase. Biochem. Pharmacol. 1992, 43, 1295-1301.
^{xiv}. Smiley, J. A.; Saleh, L. Active site probes for yeast OMP decarboxylase: Inhibition constants of UMP and thio-substituted UMP analogues and greatly reduced activity toward CMP-6-carboxylate, Bioorg. Chem. 1999, 27, 297-306.
^{xv}. Gabrielsen, B.; Kirsi, J. J.; Kwong C. D.; Carter, D. A.; Krauth, C. A.; Hanna, L. K.; Huggins, J. W.; Monath, T. P.; Kefauver, D. F.; Blough, H. A.; Rankin, J. T.; Bartz, C. M.; Huffman, J. H.; Smee, D. F.; Sidwell, R. W.; Shannon, W.

M.; Secrist, J. A. In-vitro and in-vivo antiviral (RNA) evaluation of orotidine 5′-monophosphate decarboxylase inhibitors and analogs including 6-azauridine-5′-(ethyl methoxyalaninyl)phosphate (a 5′-monophosphate prodrug). Antiviral Chem. Chemother. 1994,^{5, 209-220.}
^{xvi}. Nord, L. D.; Willis, R. C.; Smee, D. F.; Riley, T. A.; Revankar, G. R.; Robins, R. K. Inhibition of orotidylate decarboxylase by 4(5H)-oxo-1-beta-D-ribofuranosylpyrazolo[3,4-d] pyrimidine-3-thiocarboxamide (APR-TC) in B lymphoblasts. Activation by adenosine kinase, Biochem. Pharmacol. 1988, 37, 4697-4705.
^{xvii.} Smee, D. F.; McKernan, P. A.; Nord, L. D.; Willis, R. C.; Petrie, C. R.; Riley, T. M.; Revankar, G. R.; Robins, R. K.; Smith, R. A. Novel pyrazolo[3,4-d]pyrimidine nucleoside analog with broad-spectrum antiviral activity. Antimicrob. Agents. Chemother. 1987, 31, 1535-1541.
^{xviii}. Jones, M. E. Pyrimidine nucleotide biosynthesis in animals: Genes, enzymes, and regulation of UMP biosynthesis, Annu. Rev. Biochem. 1980, 49, 253-279.
^{xix}. Chen, J. J.; Jones, M. E. Effect of 6-azauridine on de novo pyrimidine biosynthesis in cultured Ehrlich ascites cells. Orotate inhibition of dihydrorotase and dihydroorotase dehydrogenase. J. Biol. Chem. 1979, 254, 4908-4914.
^{xx}. Cadman, E. C.; Dix, D. E.; Handschumacher, R. E.; Clinical, biological and biochemical effect of pyrazofurin. Cancer Res. 1978, 38, 682-698.
^{xxi}. Harris, P.; Poulsen, J. C. N.; Jensen, K. F.; Larsen, S. Substrate binding induces domain movements in orotidine 5′-monophosphate decarboxylase. J. Mol. Biol. 2002, 318, 1019-1029.
^{xxii}. Wu, N.; Mo, Y.; Gao, J.; Pai, E. F. Electrostatic stress in catalysis: structure and mechanism of the enzyme orotidine monophosphate decarboxylase. Proc. Natl. Acad. Sci. USA 2000, 97, 2017-2022. (Appendix A).
^{xxiii}. Appleby, T. C.; Kinsland, C.; Begley, T. P.; Ealick, S. E. The crystal structure and mechanism of orotidine 5′-monophosphate decarboxylase. Proc. Natl. Acad. Sci. USA 2000, 97, 2005-2010.
^{xxiv}. Miller, B. G.; Hassell, A. M.; Wolfenden, R.; Milburn, M. V.; Short, S. A. Anatomy of a proficient enzyme: the structure of orotidine 5′-monophosphate decarboxylase in the presence and absence of a potential transition state analog. Proc. Natl. Acad Sci. USA 2000, 97, 2011-2016.
^{xxv}. Lee, T. S.; Chong, L. T.; Chodera, J. D.; Kollman, P. A. An alternative explanation for the catalytic proficiency of orotidine 5′-phosphate decarboxylase. J. Am. Chem. Soc. 2001, 123, 12837-12848.
^{xxvi}. Miller, B. G.; Butterfoss, G. L.; Short, S. A.; Wolfenden, R. Role of enzyme-ribofuranosyl contacts in the ground state and transition state for orotidine 5′-phosphate decarboxylase: a role for substrate destabilization? Biochemistry 2001, 40, 6227-6232.
^{xxvii}. Warshel, A.; Strajbl, M.; Villa, J.; Florian, J. Remarkable rate enhancement of orotidine 5′-monophosphate decarboxylase is due to transition-state stabilization rather than to ground-state destabilization. Biochemistry 2000, 39, 14728-14738.
^{xxviii}. Harris, P.; Navarro Poulsen, J. C.; Jensen, K. F.; Larsen, S. Structural basis for the catalytic mechanism of a proficient enzyme: orotidine 5′-monophosphate decarboxylase. Biochemistry 2000, 39, 4217-4224.
^{xxix}. Miller, B. G.; Hassell, A. M.; Wolfenden, R.; Milburn, M. V.; Short, S. A. Anatomy of a proficient enzyme: The structure of orotidine 5′-monophosphate decarboxylase in the presence and absence of a potential transition-state analog. Proc. Natl. Acad. Sci. USA 2000, 97, 2011-2016.
^{xxx}. Dix, D. E.; Lehman, C. P.; Jakubowski, A.; Moyer, J. D.; Handschumacher, R. E. Pyrazofurin metabolism, enzyme inhibition, and resistance in L5178Y cells. Cancer Res. 1979, 39, 4485-4490.
^{xxxi}. Sant, M. E.; Lyons, S. D.; Kemp, A. J.; McClure, L. K.; Szabados, E.; Christopherson, R. I. Dual effects of pyrazofurin and 3-deazauridine upon pyrimidine and purine biosynthesis in mouse L1210 leukemia. Cancer. Res. 1989, 49, 2645-2650.
^{xxxii}. Imai, K.; Takaoka, A. Comparing antibody and small-molecule therapies for cancer. Nature Rev. Drug Discov. 2006, 6, 714-727.
^{xxxiii}. Hirota, K.; Tomishi, T.; Maki, Y.; Sajiki, H. Nucleosides & Nucleotides 1998, 17, 161-173.
^{xxxviii}. Tanaka, H.; Hayakawa, H.; Haraguchi, K.; Miyasaka, T. Introduction of an azido group to the C-6 position of uridine by the use of a 6-iodouridine derivative. Nucleosides & Nucleotides 1985, 4, 607-612.
^{xxxix}. Tanaka, H.; Hayakawa, H.; Miyasaka, T. “Umpolung” of reactivity at the C-6 position of uridine: a simple and general method for 6-substituted uridines, Tetrahedron 1982, 38, 2635-2642.
^{xi}. Sowa, T.; Ouchi, S. Facile synthesis of 5′-nucleotides by selective phosphorylation of a primary hydroxyl group of nucleosides with phosphoryl chloride. Bull. Chem. Soc. Jpn. 1975, 48, 2084-2090.
^{xii}. Ueda, T.; Yamamoto, M.; Yamane, A.; Imazawa, M.; Inoue, H. Conversion of uridine nucleotides to the 6-cyano derivatives: synthesis of orotidylic acid. Carbohyd. Nucleosides Nucleotides 1978, 5, 261-271.
^{xlii.} Poduch, E.; Bello, A. M.; Tang, S.; Fujihashi, M.; Pai, E. F.; Kotra, L. P. J. Med. Chem. 2006, 49, 4937-4935.
^{xliii}. Tanaka, H.; Hayakawa, H.; Haraguchi, K.; Miyasaka, T. Nucleosides & Nucleotides 1985, 4, 607-612.
^{xliv}. Tanaka, H.; Hayakawa, H.; Miyasaka, T. Tetrahedron 1982, 38, 2635-2642.
^{xlv}. Sowa, T.; Ouchi, S. Bull. Chem. Soc. Jpn. 1975, 48, 2084-2090.
^{xlvi}. Ueda, T; Yamamoto, M.; Yamane, A.; Imazawa, M.; Inoue, H. Carbohyd. Nucleosides Nucleotides 1978, 5, 261-271.
^{xlvii}. Tanaka, H.; Hayakawa, H.; Haraguchi, K.; Miyasaka, T. Introduction of an azido group to the C-6 position of uridine by the use of a 6-iodouridine derivative. Nucleosides Nucleotides 1985, 4, 607-612.
^{xlviii}. Tanaka, H.; Hayakawa, H.; Miyasaka, T. “Umpolung” of reactivity at the C-6 position of uridine: a simple and general method for 6-substituted uridines. Tetrahedron 1982, 38, 2635-2642.
^{xlix}. Sowa, T.; Ouchi, S. Facile synthesis of 5′-nucleotides by selective phosphorylation of a primary hydroxyl group of nucleosides with phosphoryl chloride. Bull. Chem. Soc. Jpn. 1975, 48, 2084-2090.
^{l}. Ueda, T; Yamamoto, M.; Yamane, A.; Imazawa, M.; Inoue, H. Conversion of uridine nucleotides to the 6-cyano derivatives: synthesis of orotidylic acid. Carbohyd. Nucleosides Nucleotides 1978, 5, 261-271.
^{li}. Labbe, A.; Tran, A. H.; Paige, C. J. Murine model of immune-mediated rejection of the acute lymphoblastic leukemia 70Z/3. J. Immunol. 2006, 176, 5354-5361.

1. A method of treating cancer comprising administering to a subject in need thereof an effective amount of compound selected from a compound of Formula I, tautomers thereof and pharmaceutically acceptable salts, solvates, and prodrugs thereof: 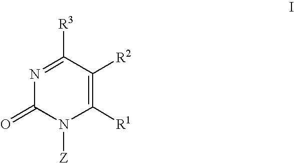
wherein,
R¹ is selected from C₁₋₆alkyl, C(O)OC₁₆alkyl, CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, H, NHC(O)OC₁-C₆alkyl and NHC(O)C₁-C₆alkyl;
Z is selected from: 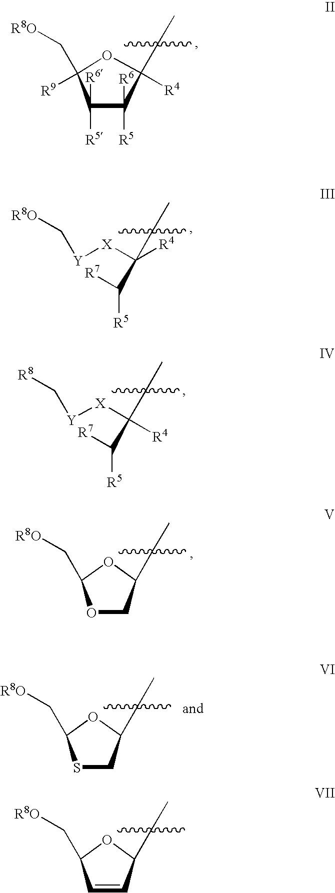
wherein,
R⁴ is selected from H, F, C₁-C₆alkyl and hydroxy-substituted-C₁-C₆alkyl;
One of R⁵ and R⁶ is selected from hydrogen and F and the other is selected from H, OH and F and one of R^{5′} and R^{6′} is selected from hydrogen and F and the other is selected from H, OH and F or R⁵ and R⁶ or R^{5′} and R^{6′} together are ═O or ═CH₂;
R⁷ is selected from H, F and OH;
R⁸ is selected from H, C(O)C₁-C₆alkyl, P(O)(OH)₂, P(O)(OC₁-C₆alkyl)₂ and P(O)(OC₁-C₆alkyl)OH;
R⁹ is selected from H, F, N₃, CN, C₁-C₆alkyl; and
X—Y is selected from —CH₂—O—, O—CH₂—, —CH₂—S— and —S—CH₂—, with the proviso that when R² is halo, in particular fluorine, R¹ is not iodo.

2. The method according to claim 1, wherein R¹ in the compounds of Formula I is selected from CH₃, CH₂CH₃, C(O)CH₃, C(O)CH₂CH₃, CN, N₃, I, Br, —CHO, —NHNH₂, —NHOH, —ONH₂, —NC, NH₂, NHCH₃, N(CH₃)₂, NHCO₂C₁₋₆alkyl, NHOH, ONH₂, C(O)NH₂.

3. The method according to claim 2, wherein R¹ in the compounds of Formula I is NH₂ or N₃.

4. (canceled)

5. The method according to claim 1, wherein R² in the compounds of Formula I is F.

6. (canceled)

7. The method according to claim 6, wherein R³ in the compounds of Formula I is OH and the compound of Formula I has the following tautomeric structure:

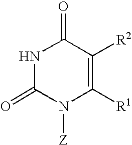

8. The method according to claim 1, wherein in the compounds of Formula I, Z is Formula II.

9. (canceled)

10. The method according to claim 1, wherein, R⁵ and R^{5′} are both OH and R⁶ and R^{6′} are both H.

11. The method according to claim 1, wherein R⁵ is H, R^{5′} is OH and R⁶ and R^{6′} are both H.

12. The method according to claim 1, wherein R⁵ and R⁶ together are ═O and R^{5′} and R^{6′} are both H.

13. The method according to claim 1, wherein R⁵ and R⁶ together are ═O and R^{5′} is OH and R^{6′} is H.

14. The method according to claim 1, wherein R⁵ and R⁶ are both F and R^{5′} and R^{6′} are both H or R^{5′} is OH and R^{6′} is H.

15. The method according to claim 1 any, wherein R⁵is H, R⁶is OH, R^{5′} is OH and R^{6′} is H.

16. The method according to claim 1, wherein one of R⁵ and R⁶ is F and the other is H and R^{5′} and R^{6′} are both H.

17-19. (canceled)

20. The method according to claim 1, wherein R⁸ in the compounds of Formula I is selected from H, C(O)CH₃, and P(O)(OH)₂.

21. The method according to claim 1, wherein R⁹ in the compounds of Formula I is H.

22. The method according to claim 1, wherein X—Y is —O—CH₂—.

23. The method according claim 1, wherein the compound of Formula I has the following structure:

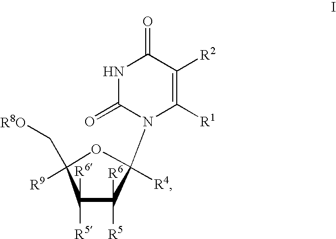

24. The method according to claim 1, wherein the cancer is selected from skin cancer, melanoma, prostate cancer, breast cancer, colorectal cancer, ovarian cancer, leukemia, lymphoma, lung cancer, head and neck cancer, espophageal cancer and pancreatic cancer.

25. (canceled)

26. The method according to claim 1, wherein the compound of Formula I is selected from:
5-fluoro-6-azido-uridine;
5-fluoro-6-azido-uridine-5′-O-monophosphate;
5-fluoro-6-amino-uridine;
5-fluoro-6-amino-uridine-5′-O-monophosphate;
5-fluoro-6-azido uridine 5′-acetate;
5-fluoro-6-azido 2′-deoxyuridine;
5-fluoro-6-azido-2′-deoxyuridine-5′-O-monophosphate;
5-fluoro-6-amino-uridine 5′-acetate;
5-fluoro-6-amino-2′-deoxyuridine;
5-fluoro-6-amino-2′-deoxyuridine-5′-O-monophosphate;
6-iodo-uridine;
6-iodo-uridine-5′-O-monophosphate;
6-iodo-uridine 5′-acetate;
6-iodo-2′-deoxyuridine;
6-iodo-2′-deoxyuridine-5′-O-monophosphate;
6-methyl-uridine;
6-methyl-uridine-5′-O-monophosphate;
6-methyl-uridine 5′-acetate;
6-methyl-2′-deoxyuridine;
6-methyl-2′-deoxyuridine-5′-O-monophosphate;
6-hydoxyamino-uridine;
6-hydroxyamino-uridine-5′-O-monophosphate;
6-hydroxyamino-uridine 5′-acetate;
6-hydroxyamino-2′deoxyuridine;
6-hydroxyamino-2′deoxyuridine-5′-O-monophosphate;
6-formyl-uridine;
6-formyl-uridine-5′-O-monophosphate;
6-formyl-uridine 5′-acetate;
6-formyl-2′deoxyuridine;
6-formyl-2′deoxyuridine-5′-O-monophosphate;
5-fluoro-6-formyl-uridine;
5-fluoro-6-formyl-uridine-5′-O-monophosphate;
5-fluoro-6-formyl-uridine 5′-acetate;
5-fluoro-6-formyl-2′deoxyuridine;
5-fluoro-6-formyl-2′deoxyuridine-5′-O-monophosphate;
5-fluoro-6-ethyl-uridine;
5-fluoro-6-ethyl-uridine-5′-O-monophosphate;
5-fluoro-6-ethyl-uridine 5′-acetate;
5-fluoro-6-ethyl-2′deoxyuridine;
5-fluoro-6-ethyl-2′deoxyuridine-5′-O-monophosphate,
and tautomers thereof and pharmaceutically acceptable salts, solvates, and prodrugs thereof.

27. The method according to claim 1, wherein the compound of Formula I is selected from:

| | |
|---|---|
| | I |
| 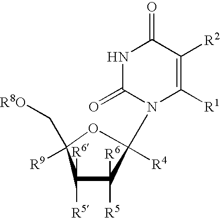 | |
| Cpd # | R¹ | R² | R⁴ | R⁵ | R⁶ | R^{5′} | R^{6′} | R⁸ | R⁹ |
|---|---|---|---|---|---|---|---|---|---|
| Ibb | NH₂ | F | H | H | OH | OH | H | H | H |
| Icc | NH₂ | F | H | H | OH | OH | H | PO₃²⁻ | H |
| Idd | N₃ | F | H | H | OH | OH | H | H | H |
| Iee | N₃ | F | H | H | OH | OH | H | PO₃²⁻ | H |
| Iff | NH₂ | F | H | F | F | OH | H | H | H |
| Igg | NH₂ | F | H | F | F | OH | H | PO₃²⁻ | H |
| Ihh | N₃ | F | H | F | F | OH | H | H | H |
| Iii | N₃ | F | H | F | F | OH | H | PO₃²⁻ | H |
| Ijj | NH₂ | F | H | F | F | H | H | H | H |
| Ikk | NH₂ | F | H | F | F | H | H | PO₃²⁻ | H |
| Ill | N₃ | F | H | F | F | H | H | H | H |
| Imm | N₃ | F | H | F | F | H | H | PO₃²⁻ | H |
| | | | | | | | | |
|---|---|---|---|---|---|---|---|---|
| Inn | NH₂ | F | H | ═O | OH | H | H | H |
| Ioo | NH₂ | F | H | ═O | OH | H | PO₃²⁻ | H |
| Ipp | N₃ | F | H | ═O | OH | H | H | H |
| Iqq | N₃ | F | H | ═O | OH | H | PO₃²⁻ | H |
| Iad | CH₃ | H | H | H | H | OH | H | H |
| Iae | NHOH | H | H | OH | H | OH | H | H |
| Iaf | CHO | H | H | OH | H | OH | H | H |
| Iag | CHO | F | H | OH | H | OH | H | H |
| Iah | C₂H₅ | F | H | OH | H | OH | H | H |

28. A compound of Formula Ia selected from: 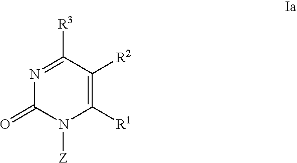
R¹ is selected from CN, N₃, I, Br, —CHO, —NHNH₂, —NHOH, —ONH₂, —NC, NH₂, NH(C₁₋₆alkyl), N(C₁₋₆alkyl)(C₁₋₆alkyl), NHCO₂C₁₋₆alkyl, NHOH, ONH₂, C(S)NH₂, C(O)NH₂;
R² is selected from H, halo, C₁-C₆alkyl, C₁-C₆alkoxy, fluoro-substituted-C₁-C₆alkyl, fluoro-substituted-C₁-C₆alkoxy, N₃, NH₂ and CN;
R³ is selected from OH, NH₂, NHC(O)OC₁-C₆alkyl and NHC(O)C₁-C₆alkyl;
Z is selected from 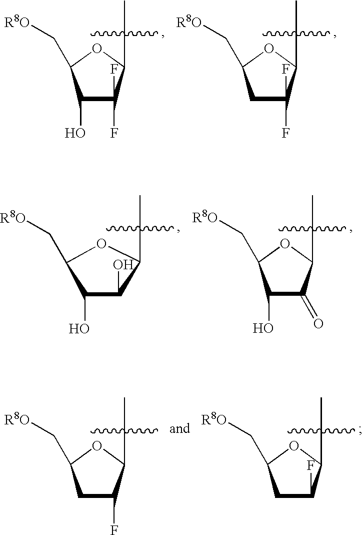
R⁸ is selected from H, C(O)C₁-C₆alkyl, P(O)(OH)₂, P(O)(OC₁-C₆alkyl)₂ and P(O)(OC₁-C₆alkyl)OH,
tautomers thereof, and pharmaceutically acceptable salts, solvates and prodrugs thereof.

29. The compound according to claim 28, wherein R³ is OH and the compound exists in the following tautomeric form:

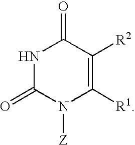

30. The compound according to claim 28, wherein R³ is selected from NH₂, NHC(O)OC₁-C₆alkyl and NHC(O)C₁-C₆alkyl, and the compound exists in the following tautomeric form: 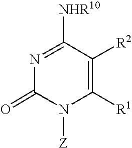
wherein R¹⁰ is selected from H, C(O)OC₁-C₆alkyl and C(O)C₁-C₆alkyl H.
